(19) United States
(12) Patent Application Publication
(10) Pub. No.: US 2009/0197853 A1
(43) Pub. Date: Aug. 6, 2009

(54) METHODS AND COMPOSITIONS OF TREATING CANCER

(75) Inventor: Darren Magda, Cupertino, CA (US)

Correspondence Address: WILSON SONSINI GOODRICH & ROSATI, 650 PAGE MILL ROAD, PALO ALTO, CA, 94304-1050, US

(73) Assignee: PHARMACYCLICS, INC., Sunnyvale, CA (US)

(21) Appl. No.: 12/090,347

(22) PCT Filed: Nov. 16, 2006
(86) PCT No.: PCT/US06/44829

§ 371 Date: Aug. 28, 2008

Related U.S. Application Data
(60) Provisional application No. 60/737,601, filed on Nov. 16, 2005.

(51) Int. Cl. A61K 31/28; C07D 487/22; A61K 31/407; C12Q 1/68; A61P 35/00

(52) U.S. Cl. 514/185; 540/471; 514/410; 435/6

(57) ABSTRACT

The present disclosure involves the use of metal-containing texaphyrins and zinc (II) reagents for the treatment of tumors, atheromas and other neoplastic tissue. The present application demonstrates increased oxidative stress, alterations in zinc homeostasis, cell cycle arrest, and apoptosis of cancer cells in the presence of texaphyrins and/or zinc. One aspect is to monitor oxidative stress and/or alterations in zinc homeostasis in target cells prior to and/or after treatment with metal-containing texaphyrins and/or zinc (II) reagents as a predictor for treatment efficacy. The present disclosure provides molecular basis for the cell cycle arrest and apoptosis on cancer cells in the presence of texaphyrins and zinc. Another aspect is to monitor different genes involved in response to treatment with texaphyrins and zinc prior to and/or after treatment as predictors for treatment efficacy.

### RELATED APPLICATIONS

This application claims benefit of U.S. Provisional Application No. 60/737,601, entitled “Methods and compositions for treating of cancer” filed on Nov. 16, 2005.

### FIELD OF THE INVENTION

Methods and compositions for treating tumors, atheromas and other neoplastic tissue as well as other conditions that are responsive to the induction of oxidative stress and/or changes in cellular zinc levels by administration of a metal-containing texaphyrin and/or a zinc (II) reagent.

### BACKGROUND OF THE INVENTION

Cancer is a serious threat to modern society. Worldwide, more than 10 million people are diagnosed with cancer every year and it is estimated that this number will grow to 15 million new cases every year by 2020. Cancer causes six million deaths every year or 12% of the deaths worldwide. Current treatment options are often limited but widely employed. Of the 1.2 million patients newly diagnosed with cancer in the United States annually, approximately 50% will be treated with radiation therapy as part of the initial disease management. Approximately 150,000 additional patients with recurrent cancer may receive radiation therapy each year in the U.S. Chemotherapy is administered to about 350,000 cancer patients in the U.S. annually. There remains a need for methods that can treat cancer. Texaphyrins are rationally designed small molecules having a ring-shaped chemical structure, usually containing one of several metal atoms. The physical and chemical characteristics of texaphyrin molecules are determined by the properties of the ring and the type of metal atom inserted into the ring. Texaphyrins are designed to selectively concentrate in diseased tissue such as tumor cells and atherosclerotic plaque inside blood vessels. Inside diseased cells, texaphyrins block crucial steps in cellular metabolism and disrupt bioenergetic processes. Texaphyrins are designed to provide a valuable therapeutic approach to a broad range of diseases. These can be used for the treatment of a variety of diseases, including cancer, atherosclerosis and cardiovascular diseases, and potentially neurodegenerative diseases, inflammatory diseases, and HIV/AIDS.

### SUMMARY OF THE INVENTION

The present application is directed to methods and compositions for treating tumors, atheromas and other neoplastic tissue as well as other conditions that are responsive to the induction of oxidative stress and/or changes in cellular zinc levels. The present application involves the use of metal-containing texaphyrins and/or zinc (II) reagents for treatment of the diseases mentioned above. The methods of the present application demonstrate increased oxidative stress, alterations of zinc homeostasis, cell cycle arrest, and apoptosis of cancer cells in the presence of texaphyrins and/or zinc. One aspect is to monitor oxidative stress and/or alterations in zinc homeostasis in plasma and in target cells prior to and/or after treatment with metal-containing texaphyrins and/or zinc (II) reagents as a predictor of treatment efficacy.

In one embodiment are compositions comprising an amount of a metal-containing texaphyrin (and in one embodiment a lanthanide-containing texaphyrin) and/or a zinc (II) reagent in an amount sufficient to change the activity or level of a biomarker. In certain embodiments, the sufficient amount is an individual sufficient amount. In a further embodiment, the individual sufficient amount is in an individual having a tumor or other neoplastic tissue. In an alternative embodiment, the sufficient amount is a population sufficient amount. In a further embodiment, the population sufficient amount is in a population having a tumor or other neoplastic tissue. In certain embodiments, the biomarker is a biomarker presented herein, including in the Examples and in the Figures.

In one embodiment are compositions comprising an amount of a metal-containing texaphyrin (and in one embodiment a lanthanide-containing texaphyrin) sufficient to cause a reduction in thioredoxin reductase activity of between about 10 to about 90%. In a further embodiment, the reduction in thioredoxin reductase activity is at least about 30%. In a further embodiment, the sufficient amount is an individual sufficient amount. In a further embodiment, the individual sufficient amount is in an individual having a tumor or other neoplastic tissue. In an alternative embodiment, the sufficient amount is a population sufficient amount. In a further embodiment, the population sufficient amount is in a population having a tumor or other neoplastic tissue.

In further embodiments of any of the aforementioned embodiments, the compositions further comprise an amount of a zinc (II) reagent sufficient to cause a reduction in thioredoxin reductase activity of between about 10 to about 90%. In further embodiments, the zinc (II) reagent is selected from the group consisting of zinc acetate, zinc chloride, zinc citrate, zinc lactate zinc gluconate, L-carnosine salt, zinc fetuin, zinc sulfate, zinc bacitracin, zinc seleno-bacitracin, chelated zinc, and zinc ionophores such as zinc 1-hydroxypyridine-2-thiol. In further embodiments, the zinc (II) reagent is zinc acetate. In further embodiments, the reduction in thioredoxin reductase activity is at least about 60%. In further embodiments, the sufficient amount is an individual sufficient amount. In further embodiments, the individual sufficient amount is in an individual having a tumor or other neoplastic tissue. In alternative embodiments, the sufficient amount is a population sufficient amount. In further embodiments, the population sufficient amount is in a population having a tumor or other neoplastic tissue.

In further embodiments of any of the aforementioned embodiments, the compositions further comprise, actinomycin D or cycloheximide.

In further embodiments of any of the aforementioned embodiments, the metal-containing texaphyrin is a compound of Formula III:

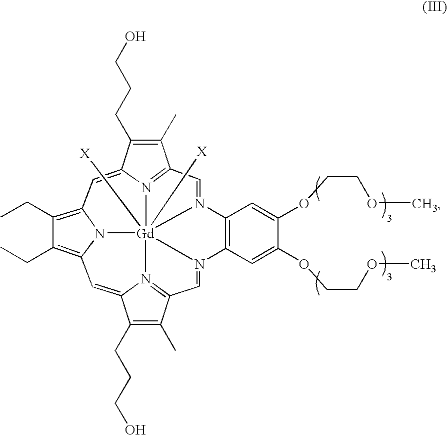

or a compound of Formula IV,

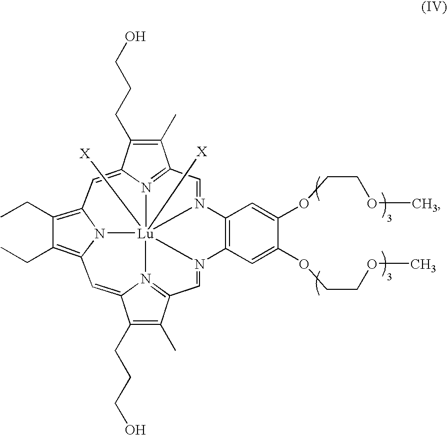

wherein X is independently selected from the group consisting of OH⁻, AcO⁻, Cl⁻, Br⁻, I⁻, F⁻, H₂PO₄⁻, ClO⁻, ClO₂⁻, ClO₃⁻, ClO₄⁻, HCO₃⁻, HSO₄⁻, NO₃⁻, N₃⁻, CN⁻, SCN⁻, OCN⁻; sugar derivatives, cholesterol derivatives, PEG acids, organic acids, organosulfates, organophosphates, phosphates or inorganic ligands; or X is derived from an acid selected from the group consisting of gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid. In further embodiments, the concentration of either the compound of Formula III or Formula IV is about 2.5 μM.

In further embodiments of any of the aforementioned embodiments, the compositions further comprise a pharmaceutically acceptable excipient. In a further embodiment, the pharmaceutically acceptable excipient is suited for intravenous administration.

In another aspect are methods for treating cancer comprising: administering to a patient having cancer an amount of a metal-containing texaphyrin (and in one embodiment a lanthanide-containing texaphyrin) sufficient to cause a reduction in thioredoxin reductase activity of between about 10 to about 90%. In a further embodiment, the reduction in thioredoxin reductase activity is at least about 30%. In a further embodiment, the sufficient amount is an individual sufficient amount. In an alternative embodiment, the sufficient amount is a population sufficient amount.

In a further embodiment of any of the aforementioned embodiments, the method further comprises administering to the patient having cancer an amount of a zinc (II) reagent sufficient to cause a reduction in thioredoxin reductase activity of between about 10 to about 90%. In a further embodiment, the zinc (II) reagent is selected from the group consisting of zinc acetate, zinc chloride, zinc citrate, zinc lactate zinc gluconate, L-carnosine salt, zinc fetuin, zinc sulfate, zinc bacitracin, zinc seleno-bacitracin, chelated zinc, and zinc ionophores such as zinc 1-hydroxypyridine-2-thiol. In a further embodiment, the zinc (II) reagent is zinc acetate. In a further embodiment, the reduction in thioredoxin reductase activity is between about 10 to 90%. In a further embodiment, the reduction in thioredoxin reductase activity is at least about 60%. In a further embodiment, the sufficient amount is an individual sufficient amount. In an alternative embodiment, the sufficient amount is a population sufficient amount.

In a further embodiment of any of the aforementioned embodiments, the method further comprises administering to the patient actinomycin D or cycloheximide.

In a further embodiment of any of the aforementioned embodiments, the metal-containing texaphyrin is a compound of Formula III:

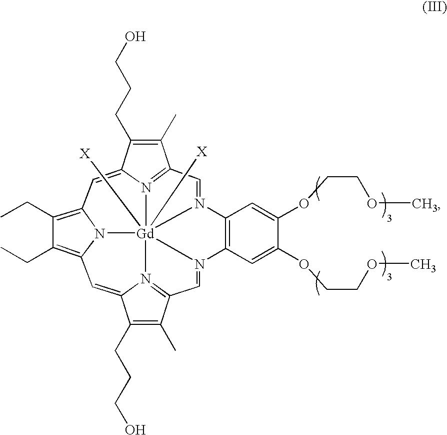

or a compound of Formula IV,

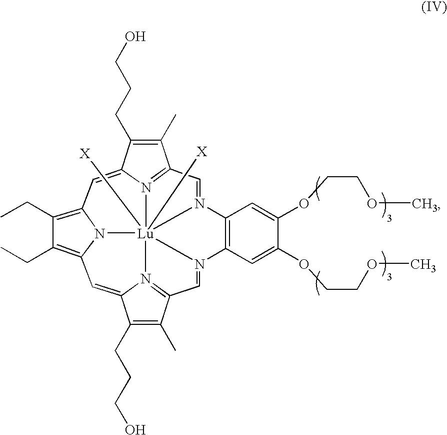

wherein X is independently selected from the group consisting of OH⁻, AcO⁻, Cl⁻, Br⁻, I⁻, F⁻, H₂PO₄⁻, ClO⁻, ClO₂⁻, ClO₃⁻, ClO₄⁻, HCO₃⁻, HSO₄⁻, NO₃⁻, N₃⁻, CN⁻, SCN⁻, OCN⁻; sugar derivatives, cholesterol derivatives, PEG acids, organic acids, organosulfates, organophosphates, phosphates or inorganic ligands; or X is derived from an acid selected from the group consisting of gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid. In further embodiments, the concentration of either the compound of Formula III or Formula IV is about 2.5 μM.

In another aspect are compositions for treating cancer comprising an amount of a metal-containing texaphyrin (and in one embodiment a lanthanide-containing texaphyrin) and an amount of a zinc (II) reagent sufficient to cause an increase in a HIF-1α level of about 3.0 fold. In a further embodiment, the sufficient amount is an individual sufficient amount. In a further embodiment, the individual sufficient amount is in an individual having a tumor or other neoplastic tissue. In an alternative embodiment, the sufficient amount is a population sufficient amount. In a farther embodiment, the population sufficient amount is in a population having a tumor or other neoplastic tissue.

In a further embodiment of any of the aforementioned embodiments, the zinc (II) reagent is selected from the group consisting of zinc acetate, zinc chloride, zinc citrate, zinc lactate zinc gluconate, L-carnosine salt, zinc fetuin, zinc sulfate, zinc bacitracin, zinc seleno-bacitracin, chelated zinc, and zinc ionophores such as zinc 1-hydroxypyridine-2-thiol.

In a farther embodiment of any of the aforementioned embodiments, the metal-containing texaphyrin is a compound of Formula III:

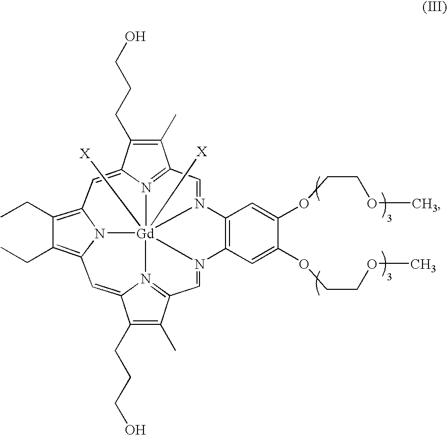

or a compound of Formula IV,

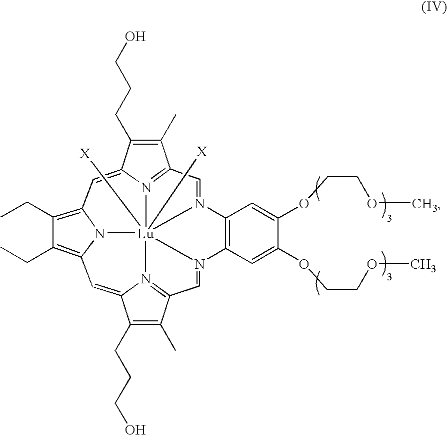

wherein X is independently selected from the group consisting of OH⁻, AcO⁻, Cl⁻, Br⁻, I⁻, F⁻, H₂PO₄⁻, ClO⁻, ClO₂⁻, ClO₃⁻, ClO₄⁻, HCO₃⁻, HSO₄⁻, NO₃⁻, N₃⁻, CN⁻, SCN⁻, OCN; sugar derivatives, cholesterol derivatives, PEG acids, organic acids, organosulfates, organophosphates, phosphates or inorganic ligands; or X is derived from an acid selected from the group consisting of gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid. In further embodiments, the concentration of either the compound of Formula III or Formula IV is about 2.5 μM.

In a further embodiment of any of the aforementioned embodiments, the composition further comprises a pharmaceutically acceptable excipient. In a further embodiment, the pharmaceutically acceptable excipient is suited for intravenous administration.

In another aspect are methods for treating cancer comprising: administering to a patient having cancer an amount of a metal-containing texaphyrin (and in one embodiment a lanthanide-containing texaphyrin) and an amount of a zinc (II) reagent sufficient to cause an increase in a HIF-1α level of about 3.0 fold. In a further embodiment, the zinc (II) reagent is selected from the group consisting of zinc acetate, zinc chloride, zinc citrate, zinc lactate zinc gluconate, L-carnosine salt, zinc fetuin, zinc sulfate, zinc bacitracin, zinc seleno-bacitracin, chelated zinc, and zinc ionophores such as zinc 1-hydroxypyridine-2-thiol. In a further embodiment, the metal-containing texaphyrin is a compound of Formula III:

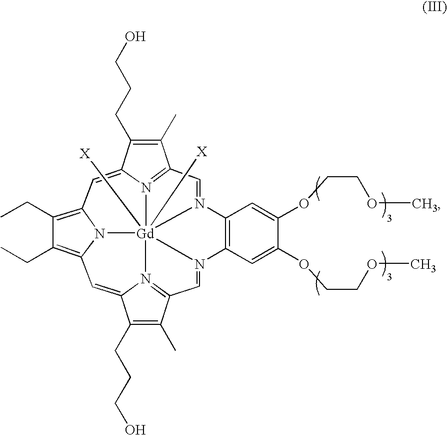

or a compound of Formula IV,

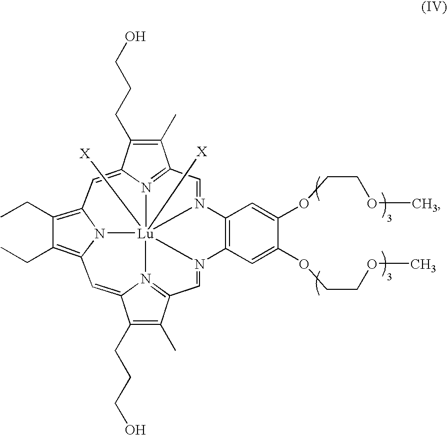

wherein X is independently selected from the group consisting of OH⁻, AcO⁻, Cl⁻, Br⁻, I⁻, F⁻, H₂PO₄⁻, ClO⁻, ClO₂⁻, ClO₃⁻, ClO₄, HCO₃⁻, HSO₄⁻, NO₃⁻, N₃⁻, CN⁻, SCN⁻, OCN⁻; sugar derivatives, cholesterol derivatives, PEG acids, organic acids, organosulfates, organophosphates, phosphates or inorganic ligands; or X is derived from an acid selected from the group consisting of gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid. In further embodiments, the concentration of either the compound of Formula III or Formula IV is about 2.5 μM.

In another embodiment, is a composition for treating cancer comprising a therapeutically effective amount of a metal-containing texaphyrin (and in one embodiment a lanthanide-containing texaphyrin) wherein the reduction in thioredoxin reductase activity is about 30%. In another embodiment is a composition for treating cancer comprising a therapeutically effective amount of a metal-containing texaphyrin (and in one embodiment a lanthanide-containing texaphyrin) and a therapeutically effective amount of a zinc (II) reagent wherein the metal-containing texaphyrin (and in one embodiment a lanthanide-containing texaphyrin) and the zinc (II) reagent causes a reduction in thioredoxin reductase activity of between about 10 to about 90%. In one embodiment is a composition for treating cancer comprising a therapeutically effective amount of a metal-containing texaphyrin (and in one embodiment a lanthanide-containing texaphyrin) and a therapeutically effective amount of a zinc (II) reagent wherein the zinc (II) reagent is selected from the group consisting of zinc acetate, zinc chloride, zinc citrate, zinc lactate zinc gluconate, L-carnosine salt, zinc fetuin, zinc sulfate, zinc bacitracin, zinc seleno-bacitracin, chelated zinc, and zinc ionophores such as zinc 1-hydroxypyridine-2-thiol wherein the reduction in thioredoxin reductase activity is between about 10 to about 90%. In another embodiment is a composition for treating cancer comprising a therapeutically effective amount of a metal-containing texaphyrin (and in one embodiment a lanthanide-containing texaphyrin) and a therapeutically effective amount of a zinc acetate wherein the reduction in thioredoxin reductase activity is between about 10 to about 90%. In a further embodiment is a composition for treating cancer comprising a therapeutically effective amount of a metal-containing texaphyrin (and in one embodiment a lanthanide-containing texaphyrin) and a therapeutically effective amount of a zinc (II) reagent wherein the reduction in thioredoxin reductase activity is between about 10 to 90%. In a further embodiment is a composition for treating cancer comprising a therapeutically effective amount of a metal-containing texaphyrin (and in one embodiment a lanthanide-containing texaphyrin) and a therapeutically effective amount of a zinc (II) reagent wherein the reduction in thioredoxin reductase activity is about 60%. In a further embodiment is a composition for treating cancer comprising a therapeutically effective amount of a metal-containing texaphyrin (and in one embodiment a lanthanide-containing texaphyrin) and a zinc (II) reagent further comprising actinomycin D or cycloheximide. In another embodiment is a composition for treating cancer comprising a metal-containing texaphyrin of Formula III

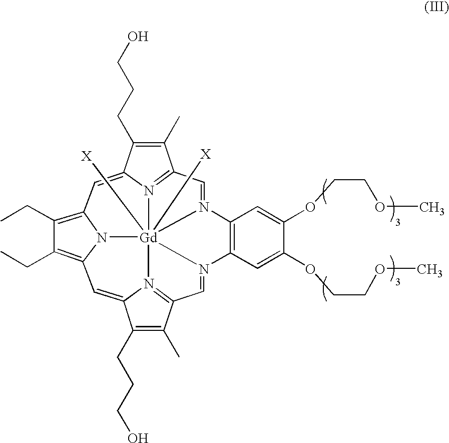

or a compound of Formula IV:

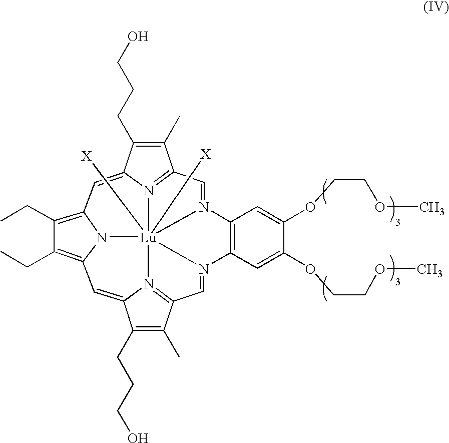

wherein a reduction in thioredoxin reductase activity is between about 10 to about 90%, and wherein X is independently selected from the group consisting of OH⁻, AcO⁻, Cl⁻, Br⁻, I⁻, F⁻, H₂PO₄⁻, ClO⁻, ClO₂⁻, ClO₃⁻, ClO₄⁻, HCO₃⁻, HSO₄⁻, NO₃⁻, N₃⁻, CN⁻, SCN⁻, OCN⁻; sugar derivatives, cholesterol derivatives, PEG acids, organic acids, organosulfates, organophosphates, phosphates or inorganic ligands; or X is derived from an acid selected from the group consisting of gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid. In a further embodiment is the composition for treating cancer comprising a therapeutically effective amount of a compound of Formula III or Formula IV having a concentration of about 2.5 μM.

In one embodiment is a method for treating cancer comprising administering a therapeutically effective amount of a metal-containing texaphyrin (and in one embodiment a lanthanide-containing texaphyrin) wherein the metal-containing texaphyrin (and in one embodiment a lanthanide-containing texaphyrin) causes a reduction in thioredoxin reductase activity of between about 10 to about 90%. In another embodiment, is a method for treating cancer comprising administering a therapeutically effective amount of a metal-containing texaphyrin (and in one embodiment a lanthanide-containing texaphyrin) wherein the reduction in thioredoxin reductase activity is about 30%. In one embodiment is a method for treating cancer comprising administering a therapeutically effective amount of a metal-containing texaphyrin (and in one embodiment a lanthanide-containing texaphyrin) and a therapeutically effective amount of a zinc (II) reagent wherein a reduction in thioredoxin reductase activity is between about 10 to about 90%. In another embodiment is a method treating cancer comprising administering a therapeutically effective amount of a metal-containing texaphyrin (and in one embodiment a lanthanide-containing texaphyrin) and a therapeutically effective amount of a zinc (II) reagent wherein the zinc (II) reagent is selected from the group consisting of zinc acetate, zinc chloride, zinc citrate, zinc lactate zinc gluconate, L-carnosine salt, zinc fetuin, zinc sulfate, zinc bacitracin, zinc seleno-bacitracin, chelated zinc, and zinc ionophores such as zinc 1-hydroxypyridine-2-thiol wherein a reduction in thioredoxin reductase activity is between about 10 to about 90%. In another embodiment is a method for treating cancer comprising administering a therapeutically effective amount of a metal-containing texaphyrin (and in one embodiment a lanthanide-containing texaphyrin) and a therapeutically effective amount of a zinc acetate wherein a reduction in thioredoxin reductase activity is between about 10 to about 90%. In another embodiment is a method for treating cancer comprising administering a therapeutically effective amount of a metal-containing texaphyrin (and in one embodiment a lanthanide-containing texaphyrin) and a therapeutically effective amount of a zinc (II) reagent wherein the reduction in thioredoxin reductase activity is between about 10 to 90%. In a further embodiment is a method for treating cancer comprising administering a therapeutically effective amount of a metal-containing texaphyrin (and in one embodiment a lanthanide-containing texaphyrin) and a therapeutically effective amount of a zinc (II) reagent wherein the reduction in thioredoxin reductase activity is about 60%. In a further embodiment is a method for treating cancer comprising administering a therapeutically effective amount of a metal-containing texaphyrin (and in one embodiment a lanthanide-containing texaphyrin) and a zinc (II) reagent further comprising actinomycin D or cycloheximide. In another embodiment is the method for treating cancer comprising administering a metal-containing texaphyrin of Formula III

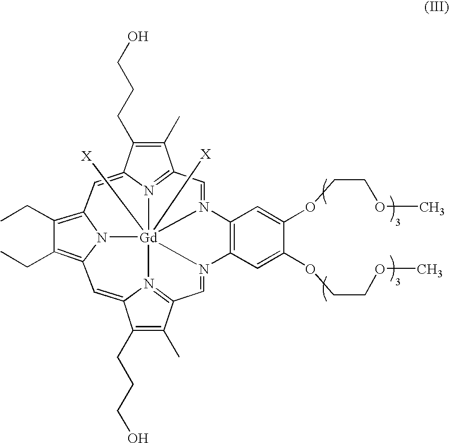

or a compound of Formula IV:

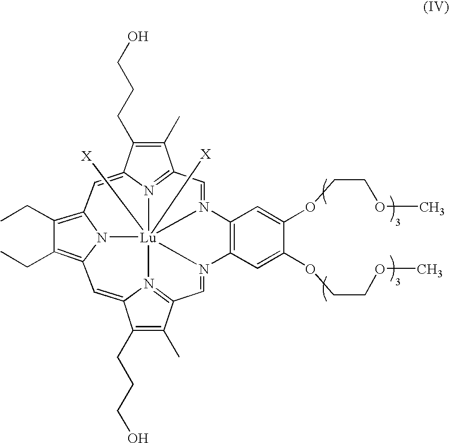

wherein the metal-containing texaphyrin (and in one embodiment a lanthanide-containing texaphyrin) causes a reduction in thioredoxin reductase activity of between about 10 to about 90%. In a further embodiment is the method for treating cancer comprising administering a therapeutically effective amount of a compound of Formula III or Formula IV having a concentration of about 2.5 μM.

In one aspect is a composition for treating cancer comprising a therapeutically effective amount of a metal-containing texaphyrin (and in one embodiment a lanthanide-containing texaphyrin) and a therapeutically effective amount of a zinc (II) reagent wherein the metal-containing texaphyrin (and in one embodiment a lanthanide-containing texaphyrin) and the zinc (II) reagent causes between about a 1.5 to about a 1.8 fold increase in a dichlorofluorescein level. In one embodiment is a composition for treating cancer comprising a therapeutically effective amount of a metal-containing texaphyrin (and in one embodiment a lanthanide-containing texaphyrin) and a therapeutically effective amount of a zinc (II) reagent wherein the metal-containing texaphyrin is a compound of Formula III:

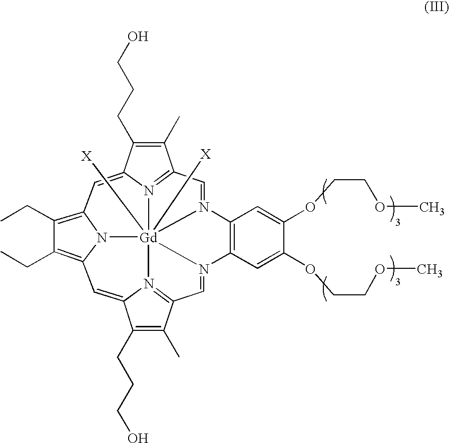

or a compound of Formula IV:

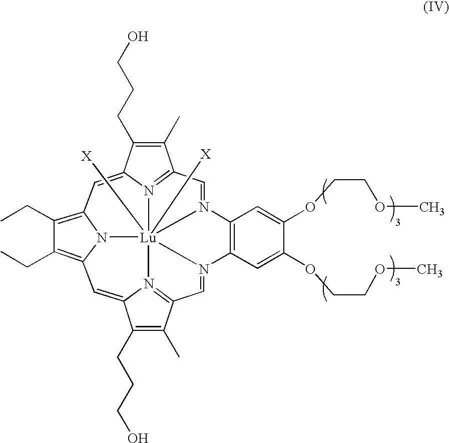

wherein a therapeutically effective amount of a metal-containing texaphyrin (and in one embodiment a lanthanide-containing texaphyrin) and a therapeutically effective amount of a zinc (II) reagent causes between about a 1.5 to about a 1.8 fold increase in a dichlorofluorescein level.

In one aspect is a method for treating cancer comprising administering a therapeutically effective amount of a metal-containing texaphyrin (and in one embodiment a lanthanide-containing texaphyrin) and a therapeutically effective amount of a zinc (II) reagent wherein the metal-containing texaphyrin (and in one embodiment a lanthanide-containing texaphyrin) and the zinc (II) reagent causes between about a 1.5 to about a 1.8 fold increase in a dichlorofluorescein level. In one embodiment is a method for treating cancer comprising administering a therapeutically effective amount of a metal-containing texaphyrin (and in one embodiment a lanthanide-containing texaphyrin) and a therapeutically effective amount of a zinc (II) reagent wherein the metal-containing texaphyrin (and in one embodiment a lanthanide-containing texaphyrin) and the zinc (II) reagent causes between about a 1.5 to about a 1.8 fold increase in a dichlorofluorescein level and wherein the metal-containing texaphyrin is a compound of Formula III:

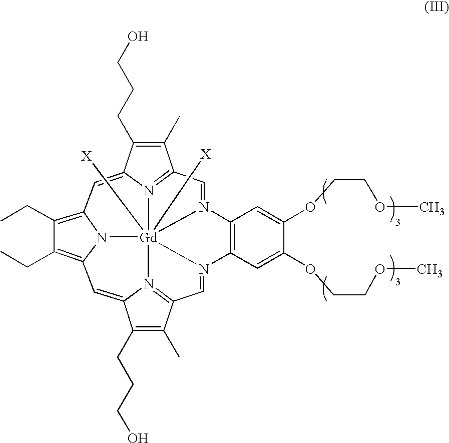

or a compound of Formula IV:

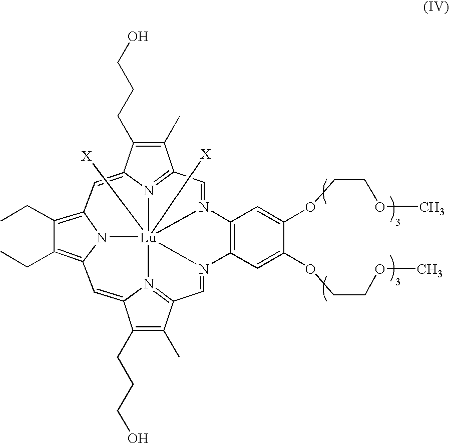

wherein the metal-containing texaphyrin (and in one embodiment a lanthanide-containing texaphyrin) and the zinc (II) reagent causes between about a 1.5 to about a 1.8 fold increase in a dichlorofluorescein level.

In one embodiment is a composition for treating cancer comprising a therapeutically effective amount of a metal-containing texaphyrin (and in one embodiment a lanthanide-containing texaphyrin) and a therapeutically effective amount of a zinc (II) reagent wherein the metal-containing texaphyrin (and in one embodiment a lanthanide-containing texaphyrin) and the zinc (II) reagent causes an increase in a HIF-1α level of about 3.0 fold. In another embodiment is a composition for treating cancer wherein the metal-containing texaphyrin is a compound of Formula III:

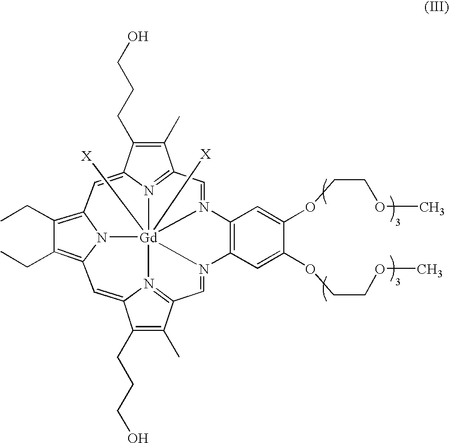

or a compound of Formula IV:

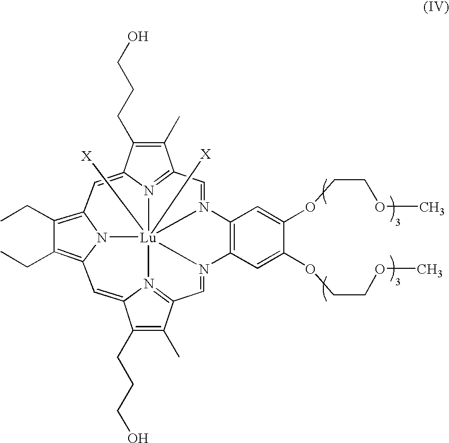

wherein the metal-containing texaphyrin (and in one embodiment a lanthanide-containing texaphyrin) and the zinc (II) reagent causes an increase in a HIF-1α level of about 3.0 fold.

In one embodiment is a method for treating cancer comprising administering a therapeutically effective amount of a metal-containing texaphyrin (and in one embodiment a lanthanide-containing texaphyrin) and a therapeutically effective amount of a zinc (II) reagent wherein the metal-containing texaphyrin (and in one embodiment a lanthanide-containing texaphyrin) and the zinc (II) reagent causes an increase in a HIF-1α level of about 3.0 fold. In another embodiment is a method for treating cancer comprising administering a therapeutically effective amount of a metal-containing texaphyrin (and in one embodiment a lanthanide-containing texaphyrin) and a therapeutically effective amount of a zinc (II) reagent wherein the metal-containing texaphyrin is a compound of Formula III:

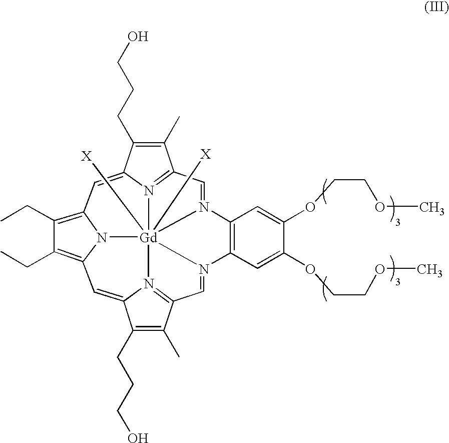

or a compound of Formula IV:

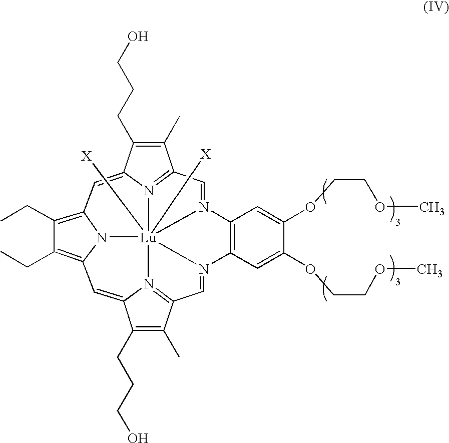

wherein the metal-containing texaphyrin (and in one embodiment a lanthanide-containing texaphyrin) and the zinc (II) reagent causes an increase in a HIF-1α level of about 3.0 fold.

One embodiment involves a method for predicting treatment efficacy by monitoring oxidative stress in plasma and in target cells of an animal subject bearing a tumor, atheroma or a neoplastic disease prior to and/or after treatment with a metal-containing texaphyrin (and in one embodiment a lanthanide-containing texaphyrin) and/or a zinc (II) reagent. In another embodiment, the monitoring of oxidative stress in plasma and in target cells is used to modulate the treatment with a metal-containing texaphyrin (and in one embodiment a lanthanide-containing texaphyrin) and/or a zinc (II) reagent. Another embodiment involves a method for predicting treatment efficacy by monitoring oxidative stress related genes in plasma and in target cells of an animal subject bearing a tumor, atheroma or a neoplastic disease prior to and/or after treatment with a metal-containing texaphyrin (and in one embodiment a lanthanide-containing texaphyrin) and/or a zinc (II) reagent. In yet another embodiment, the monitoring of oxidative stress related genes in plasma and in target cells is used to modulate the administration of said treatment with a metal-containing texaphyrin (and in one embodiment a lanthanide-containing texaphyrin) and/or a zinc (II) reagent.

One embodiment involves a method for monitoring intracellular levels of zinc in plasma and in target cells of an animal subject bearing a tumor, atheroma or other neoplastic disease prior and/or after treatment with a metal-containing texaphyrin (and in one embodiment a lanthanide-containing texaphyrin) and/or a zinc (II) reagent. In another embodiment, the monitoring of intracellular levels of zinc is used to predict treatment efficacy, modulate the administration of treatment and/or consider other alternative treatments. Another embodiment involves a method for predicting treatment efficacy by monitoring zinc related genes in plasma and in target cells of an animal subject bearing a tumor, atheroma or a neoplastic disease prior to and/or after treatment with a metal-containing texaphyrin (and in one embodiment a lanthanide-containing texaphyrin) and/or a zinc (II) reagent. In yet another embodiment, the monitoring of zinc related genes is used to modulate the administration of treatment with a metal-containing texaphyrin (and in one embodiment a lanthanide-containing texaphyrin) and a zinc (II) reagent.

Another aspect provides molecular basis for the cell cycle arrest and apoptosis on cancer cells in the presence of a texaphyrins and/or zinc. Another aspect is to monitor different genes involved in response to treatment with texaphyrins and zinc prior to and/or after treatment as predictors for treatment efficacy.

Another aspect is to monitor oxidative stress, alterations in zinc homeostasis and/or expression of different genes as predictors for treatment efficacy of compounds that induce the same cellular mechanisms in plasma and in target cells as metal-containing texaphyrin (and in one embodiment a lanthanide-containing texaphyrin)s and/or zinc (II) reagents

### INCORPORATION BY REFERENCE

Unless stated otherwise, all publications and patent applications mentioned in this specification are herein incorporated by reference to the same extent as if each individual publication or patent application was specifically and individually indicated to be incorporated by reference.

### BRIEF DESCRIPTION OF THE FIGURES

A better understanding of the features and advantages of the methods and compositions that are described herein may be obtained by reference to the following detailed description that sets forth illustrative embodiments, and the accompanying drawings of which:

FIG. 1A depicts the chemical structure of MGd.

FIG. 1B depicts transcript levels of metallothionein family members and ZnT1 on A549 cells treated for 4 hours with control vehicle, gadolinium acetate, or MGd as determined by Northern hybridization (top) or microarrays (bottom).

FIG. 1C depicts a schematic diagram of metallothionein gene regulation in response to oxidative stress.

FIG. 2A depicts cell viability as measured by propidium iodide exclusion.

FIG. 2B depicts transcript levels of metallothionein family members in A549 cultures treated with MGd and metal cations as determined by Northern blot analysis.

FIG. 2C depicts measurement of intracellular free zinc in A549 cells. FIG. 2C inset depicts intracellular free zinc in cells treated with MGd in serum-free medium in the absence and presence of actinomycin D.

FIG. 2D depicts the effect of MGd and zinc treatment on proliferation of A549 cells.

FIG. 3A depicts cell viability as measured by propidium iodide exclusion.

FIG. 3B depicts transcript levels of metallothionein family members and ZnT1 in PC3 cultures treated with MGd and zinc acetate for 4 hours as determined by Northern blot analysis.

FIG. 3C depicts measurement of intracellular free zinc in PC3 cells treated with MGd and zinc acetate.

FIG. 3D depicts the effect of MGd and zinc treatment on proliferation of PC3 cells.

FIG. 4A depicts viability of Ramos cells treated with zinc acetate and MGd.

FIG. 4B depicts metallothionein family member and ZnT1 RNA transcript levels of Ramos cells treated with zinc acetate and MGd.

FIG. 4C depicts FluoZn-3 fluorescence of Ramos cells treated with zinc acetate and MGd.

FIG. 4D depicts proliferation of Ramos cells treated with zinc acetate and MGd.

FIG. 5A depicts Lipoate reduction in A549 and PC3 cells treated with zinc acetate and MGd for two hours.

FIG. 5B depicts Lipoate reduction in A549 and PC3 cells treated with zinc acetate and MGd for four hours.

FIG. 5C depicts Lipoate reduction in A549 and PC3 cells treated with zinc acetate, MGd and actinomycin D for four hours.

FIG. 5D depicts Lipoate reduction in A549 and PC3 cells treated zinc 1-hydroxy-2-pyridinethione and MGd for four hours.

FIG. 5E depicts Lipoate reduction in A549 and PC3 cells treated with zinc acetate and MGd for two hours.

FIG. 5F depicts Lipoate reduction in A549 and PC3 cells treated with zinc acetate and MGd for three hours.

FIG. 5G depicts Lipoate reduction in A549 and PC3 cells treated with zinc acetate, MGd and actinomycin D for three hours.

FIG. 5H depicts Lipoate reduction in A549 and PC3 cells treated zinc 1-hydroxy-2-pyridinethione and MGd for four hours.

FIG. 6A depicts fold increase of FluoZin-3 fluorescence in live-gated cells after treatment with control vehicle (Mannitol), zinc acetate, MGd, or the combination for up to 24 hours.

FIG. 6B depicts fold increase dichlorofluorescein (DCF) fluorescence in live-gated cells after treatment with control vehicle (Mannitol), zinc acetate, MGd, or the combination for up to 24 hours.

FIG. 6C depicts percentage of live-gated cells exhibiting green (non-aggregated) JC-1 fluorescence characteristic of lost mitochondrial membrane potential after treatment with control vehicle (Mannitol), zinc acetate, MGd, or the combination for up to 24 hours.

FIG. 7 depicts DNA synthesis on Ramos cells after treatment with MGd and zinc for up to 8 hours.

FIG. 8A depicts fold increase dichlorofluorescein (DCF) fluorescence in live-gated cells after 4 hours of treatment with control vehicle (Mannitol), zinc acetate, MGd, or the combination.

FIG. 8B depicts fold increase dichlorofluorescein (DCF) fluorescence in live-gated cells after 4 hours of treatment with control vehicle (Mannitol), zinc acetate, MGd, or the combination.

FIG. 8C depicts percentage of Annexin-V stained cells after 24 and 48 hours of treatment with control vehicle (Mannitol), zinc acetate, MGd, or the combination.

FIG. 9A depicts a Venn diagram showing relationship between transcripts significantly altered by treatment with 10 μM MGd or 50 μM zinc.

FIG. 9B depicts a Venn diagram showing relationship between transcripts significantly altered by treatment with 10 μM MGd and 25 μM or 50 μM zinc.

FIG. 9C depicts the Levels of HIF-1α protein relative to control vehicle after 4 hours treatment with control vehicle (Mannitol), zinc acetate, MGd, or the combination as measured by ELISA.

FIG. 9D depicts a representative Western blots showing levels of heme oxygenase 1 (HO1) and metallothioneins 1 and 2 (MT) after 16 hours treatment with control vehicle (Mannitol), zinc acetate, MGd, or the combination.

FIG. 10 depicts differential gene regulation in response to MGd treatments

FIG. 11 depicts genes differentially expressed in response to MGd treatment.

FIG. 12 depicts transcriptional responses of stress-related genes in Ramos Cell co-treated with MGd and Zinc.

FIG. 13 depicts selected genes related to apoptosis and cell cycle control.

### DETAILED DESCRIPTION OF THE INVENTION

While embodiments have been shown and described herein, it will be obvious to those skilled in the art that such embodiments are provided by way of example only. Numerous variations, changes, and substitutions will now occur to those skilled in the art without departing from what is presently provided. It should be understood that various alternatives to the embodiments described herein may be employed in practicing what is claimed in the application. It is intended that the following claims define the scope of the application and that methods and structures within the scope of these claims and their equivalents be covered thereby.

The present application is directed to methods for treating tumors, atheromas and other neoplastic tissue as well as other conditions that are responsive to the induction of targeted oxidative stress and/or changes in cellular zinc levels. The present application involves the use of metal-containing texaphyrin (and in one embodiment a lanthanide-containing texaphyrin)s and zinc (II) reagents for treatment of the diseases mentioned above. The application demonstrates increased oxidative stress, alterations in zinc homeostasis, cell cycle arrest, and apoptosis of cancer cells in the presence of texaphyrins and zinc. One aspect is to monitor oxidative stress and/or alterations in zinc homeostasis in plasma and in target cells prior to and after treatment with metal-containing texaphyrin (and in one embodiment a lanthanide-containing [symbol] texaphyrin)s and zinc (II) reagents as a predictor for treatment efficacy. Another aspect provides molecular basis for the cell cycle arrest and apoptosis on cancer cells in the presence of a texaphyrins and zinc. Another aspect is to monitor different genes involved in response to treatment with texaphyrins and zinc prior to and after treatment as predictors for treatment efficacy. Another aspect is to monitor oxidative stress, alterations in zinc homeostasis and/or expression of different genes as predictors for treatment efficacy of compounds that induce the same cellular mechanisms in plasma and in target cells as metal-containing texaphyrin (and in one embodiment a lanthanide-containing texaphyrin)s and/or zinc (II) reagents.

### DEFINITIONS AND GENERAL PARAMETERS

As used in the present specification, the following words and phrases are generally intended to have the meanings as set forth below, except to the extent that the context in which they are used indicates otherwise.

Except as otherwise specified “neutron(s)” refer to “slow” or “thermal” neutrons of the type employed in neutron capture therapy.

The term “metal-containing texaphyrin” is intended to encompass the metallotexaphyrins of the application as disclosed, coordination complexes of the compounds of Formula I, and/or the pharmaceutically acceptable salts of such compounds.

The term “effective amount” or “therapeutically effective amount” refers to that amount of a compound of Formula I that is sufficient to effect treatment, as defined below, when administered to a mammal in need of such treatment. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The term also applies to a dose that will induce a particular response in plasma and in target cells, i.e. increase in intracellular levels of zinc. The specific dose will vary depending on the particular compound of Formula I chosen, the dosing regimen to be followed, timing of administration, the tissue to be imaged, and the physical delivery system in which it is carried.

The term “individual sufficient amount” refers to an amount in an individual receiving the compositions or methods described herein. By way of example only, an individual sufficient amount of a metal-containing texaphyrin (and in one embodiment a lanthanide-containing texaphyrin) to cause a reduction in thioredoxin reductase activity of between about 10 to about 90% means an amount of a metal-containing texaphyrin (and in one embodiment a lanthanide-containing texaphyrin) that causes a reduction in thioredoxin activity of between about 10 to about 90% in that individual. In general, after the metal-containing texaphyrin (and in one embodiment a lanthanide-containing texaphyrin) has been administered to the individual, the thioredoxin activity is determined in the individual. The activity can be determined at one time point or at several time points; in any case, an individual sufficient amount decreases the activity of thioredoxin reductase between about 10 to about 90% in that individual.

The term “population sufficient amount” refers to an amount provided to an individual wherein the amount has been statistically demonstrated in a population to achieve the desired effect; that is, the effect may not be actually observed in the individual, but it has been statistically demonstrated in a human population. By statistically demonstrated in a human population is meant that a clinical study has shown, with a p value less than 0.5, a correlation between a desired effect and an amount of agent (metal-containing texaphyrin and/or zinc (II) reagent). A prospective or retrospective study is sufficient clinical study, as is an unblinded, blinded or double-blinded clinical study. By way of example only, a population sufficient amount of a metal-containing texaphyrin (and in one embodiment a lanthanide-containing texaphyrin) to cause a reduction in thioredoxin reductase activity of between about 10 to about 90% means an amount of a metal-containing texaphyrin (and in one embodiment a lanthanide-containing texaphyrin) that has been statistically demonstrated to cause a reduction in thioredoxin activity of between about 10 to about 90% in a human population.

“Optional” or “optionally” means that the subsequently described event or circumstance may or may not occur, and that the description includes instances where said event or circumstance occurs and instances in which it does not.

The term “treatment” or “treating” means any treatment of a disease in a mammal, including: (i) preventing the disease, that is, causing the clinical symptoms of the disease not to develop; (ii) inhibiting the disease, that is, arresting the development of clinical symptoms; and/or (iii) relieving the disease, that is, causing the regression of clinical symptoms.

The term “modulation” of administration can include, e.g., administering another therapeutic agent in addition to the metal-containing texaphyrin (and in one embodiment a lanthanide-containing texaphyrin) and the zinc (II) reagent, adjusting the dosage of the metal-containing texaphyrin (and in one embodiment a lanthanide-containing texaphyrin) and/or the zinc (II) reagent, route of administration of the metal-containing texaphyrin (and in one embodiment a lanthanide-containing texaphyrin) and/or the zinc (II) reagent, frequency of administration of the metal-containing texaphyrin (and in one embodiment a lanthanide-containing texaphyrin) and/or the zinc (MD reagent, type of carrier of the metal-containing texaphyrin (and in one embodiment a lanthanide-containing texaphyrin) and/or the zinc (II) reagent, duration of treatment with the metal-containing texaphyrin (and in one embodiment a lanthanide-containing texaphyrin) and/or the zinc (II) reagent, enantiomeric form of the metal-containing texaphyrin (and in one embodiment a lanthanide-containing texaphyrin) and/or the zinc (II) reagent, crystal form of the metal-containing texaphyrin (and in one embodiment a lanthanide-containing texaphyrin) and/or the zinc (II) reagent, administering a fragment, analog, or variant of the metal-containing texaphyrin (and in one embodiment a lanthanide-containing texaphyrin) and the zinc (II) reagent or a combination thereof.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “animal subject” as used herein includes humans as well as other mammals.

### Texaphyrins

Texaphyrins are aromatic pentadentate macrocyclic “expanded porphyrins” which are considered as being an aromatic benzannulene containing both 18π and 22 π-electron delocalization pathways. Such texaphyrins and their synthesis are well known in the art. Texaphyrins and water-soluble texaphyrins, method of preparation and various uses have been described in U.S. Pat. Nos. 4,935,498, 5,162,509, 5,252,720, 5,256,399, 5,272,142, 5,292,414, 5,369,101, 5,432,171, 5,439,570, 5,451,576, 5,457,183, 5,475,104, 5,504,205, 5,525,325, 5,559,207, 5,565,552, 5,567,687, 5,569,759, 5,580,543, 5,583,220, 5,587,371, 5,587,463, 5,591,422, 5,594,136, 5,595,726, 5,599,923, 5,599,928, 5,601,802, 5,607,924, 5,622,946, and 5,714,328; PCT publications WO 90/10633, 94/29316, 95/10307, 95/21845, 96/09315, 96/40253, 96/38461, 97/26915, 97/35617, 97/46262, and 98/07733; allowed U.S. patent application Ser. Nos. 08/458,347, 08/591,318, and 08/914,272; and pending U.S. patent application Ser. Nos. 08/763,451, 08/903,099, 08/946,435, 08/975,090, 08/975,522, 08/988,336, and 08/975,526; each previously incorporated herein by reference.

Particularly texaphyrins include those represented by Formula IA:

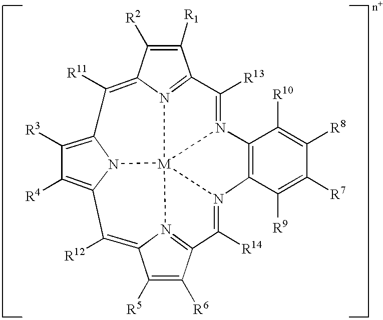

### or Formula 1B:

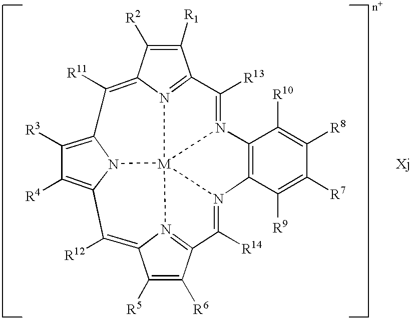

where j is 1, 2, or 3; and each X is independently selected from the group consisting of OH⁻, AcO⁻, Cl⁻, Br⁻, I⁻, F⁻, H₂PO₄⁻, ClO⁻, ClO₂⁻, ClO₃⁻, ClO₄⁻, HCO₃⁻, HSO₄⁻, NO₃⁻, N₃⁻, CN⁻, SCN⁻, and OCN⁻. In a further or alternate embodiment, each X is selected from the group consisting of sugar derivatives, cholesterol derivatives, PEG acids, organic acids, organosulfates, organophosphates, phosphates or inorganic ligands. In a further or alternate embodiment, X is derived from an acid selected from the group consisting of gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid; wherein M is a divalent metal cation or a trivalent metal cation;
R¹, R², R³, R⁴, R⁵, R⁶, R⁷, and R⁸ are independently chosen from the group consisting of hydrogen, halogen, hydroxyl, optionally substituted alkyl, optionally substituted alkenyl, optionally substituted alkynyl, optionally substituted aryl, optionally substituted heteroaryl, optionally substituted haloalkyl; nitro, acyl, optionally substituted alkoxy, saccharide, optionally substituted amino, carboxyl, optionally substituted carboxyalkyl, optionally substituted carboxyamide, optionally substituted carboxyamidealkyl, optionally substituted heterocycle, optionally substituted cycloalkyl, optionally substituted arylalkyl, optionally substituted heteroarylalkyl, and optionally substituted heterocycloalkylalkyl; and
R⁹, R¹⁰, R¹¹, R¹², R¹³ and R¹⁴ are independently hydrogen, optionally substituted alkyl, optionally substituted aryl, optionally substituted alkoxy, optionally substituted carboxyalkyl, or optionally substituted carboxyamidealkyl; with the proviso that the halogen is other than iodide and the haloalkyl is other than iodoalkyl; and
the charge, n, is an integer having a value less than or equal to 5.

The divalent or trivalent metal M is selected from the group consisting of Ca(II), Mn(II), Co(II), Ni(II), Zn(II), Cd(II), Hg(II), Fe(II), Sm(II), UO.sub.2 (II), Mn(III), Co(III), Ni(III), Fe(III), Ho(III), Ce(II), Y(III), In(III), Pr(III), Nd(III), Sm(III), Eu(III), Gd(III), Tb(III), Dy(III), Er(III), Tm(III), Yb(III), Lu(III), La(III), and U(III).

Particular texaphyrin compounds are represented by Formula IIA:

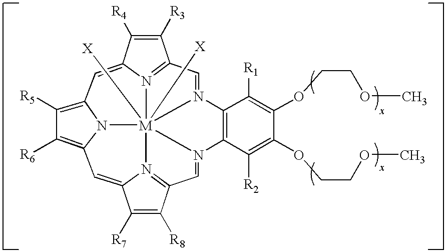

wherein

A. M is Gd(III);

B. M is Dy(III);

C. M is Y(III);

D. M is Lu(III);

E. M is Co(II);

F. M is Fe(III);

G. M is Eu(III);

H. M is Sm(III);

where j is 1, 2, or 3, and X is independently selected from the group consisting of OH⁻, AcO⁻, Cl⁻, Br⁻, I⁻, F⁻, H₂PO₄⁻, ClO⁻, ClO₂⁻, ClO₃⁻, ClO₄⁻, HCO₃⁻, HSO₄⁻, NO₃⁻, N₃⁻, CN⁻, SCN⁻, and OCN⁻; R₃, R₄, R₅, R₆, R₇ and R₈ are independently H, OH, CₙH₍₂ₙ₊₁₎O_{y} or OCₙH₍₂ₙ₊₁₎O_{y} and R₁, R₂ are independently H or C₁-C₆ alkyl where at least one of R₃, R₄, R₅, R₆, R₇ and R₈ is CₙH₍₂ₙ₊₁₎O_{y} or OCₙH₍₂ₙ₊₁₎O_{y}, having at least one hydroxyl substituent; n is a positive integer from 1 to 11; y is zero or a positive integer less than or equal to n; each x is independently selected from the group consisting of 2, 3, 4, 5, and 6; wherein at least about 98.4% of compounds of Formula II in the composition have the same structure. In one embodiment, M is Gd⁺³. In one embodiment, R₄ and R₇ are C₃H₆OH; R₅ and R₆ are C₂H₅; R₃ and R₈ are CH₃; R₁ and R₂ are H. In one embodiment, each x is 3. In one embodiment, each X is AcO⁻. In another embodiment, M is Lu⁺³. In one embodiment, R₄ and R₇ are C₃H₆OH; R₅ and R₆ are C₂H₅; R₃ and R₈ are CH₃; R₁ and R₂ are H. In one embodiment, each x is 3. In one embodiment, each X is AcO⁻. In a further embodiment, each X is selected from the group consisting of sugar derivatives, cholesterol derivatives, PEG acids, organic acids, organosulfates, organophosphates, phosphates or inorganic ligands. In a further embodiment, X is derived from an acid selected from the group consisting of gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid.

The term “alkyl” refers to a monoradical branched or unbranched saturated hydrocarbon chain having from 1 to 20 carbon atoms, or 1 to 10 carbon atoms, or 1 to 6 carbon atoms. This term is exemplified by groups such as methyl, ethyl, n-propyl, iso-propyl, n-butyl, iso-butyl, n-hexyl, n-decyl, tetradecyl, and the like.

The term “substituted alkyl” refers to an alkyl group as defined above, having from 1 to 5 substituents, 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl; or an alkyl group as defined above that is interrupted by 1-20 atoms independently chosen from oxygen, sulfur and NR^{a}, where R^{a} is chosen from hydrogen, or optionally substituted alkyl, cycloalkyl, alkenyl, cycloalkenyl, alkenyl, cycloalkenyl, alkynyl, aryl, heteroaryl and heterocyclic; or an alkyl group as defined above that has both from 1 to 5 substituents as defined above and is also interrupted by 1-20 atoms as defined above.

Another alkyl substituent is hydroxy, exemplified by hydroxyalkyl groups, such as 2-hydroxyethyl, 3-hydroxypropyl, 3-hydroxybutyl, 4-hydroxybutyl, and the like; dihydroxyalkyl groups (glycols), such as 2,3-dihydroxypropyl, 3,4-dihydroxybutyl, 2,4-dihydroxybutyl, and the like; and those compounds known as polyethylene glycols, polypropylene glycols and polybutylene glycols, and the like.

The term “alkylene” refers to a diradical of a branched or unbranched saturated hydrocarbon chain, having from 1 to 20 carbon atoms, or 1-10 carbon atoms, or 1-6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

The term “substituted alkylene” refers to: an alkylene group as defined above having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyacylamino, azido, cyano, halogen, hydroxyl, keto, thioketo, carboxyl, carboxylalkyl, thiol, thioalkoxy, substituted thioalkoxy, aryl, aryloxy, thioaryloxy, heteroaryl, heteroaryloxy, thioheteroaryloxy, heterocyclic, heterocyclooxy, thioheterocyclooxy, nitro, and —NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic. Additionally, such substituted alkylene groups include those where two substituents on the alkylene group are fused to form one or more cycloalkyl, substituted cycloalkyl, cycloalkenyl, substituted cycloalkenyl, aryl, heterocyclic or heteroaryl groups fused to the alkylene group; or an alkylene group as defined above that is interrupted by 1-20 atoms independently chosen from oxygen, sulfur and NR^{a}—, where R^{a} is chosen from hydrogen, optionally substituted alkyl, cycloalkyl, alkenyl, cycloalkenyl, alkenyl, cycloalkenyl, alkynyl, aryl, heteroaryl and heterocyclic, or groups selected from carbonyl, carboxyester, carboxyamide and sulfonyl; or an alkylene group as defined above that has both from 1 to 5 substituents as defined above and is also interrupted by 1-20 atoms as defined above.

Examples of substituted alkylenes are chloromethylene (—CH(Cl)—), aminoethylene (—CH(NH₂)CH₂—), 2-carboxypropylene isomers (—CH₂CH(CH₂H)CH₂—), ethoxyethyl (—CH₂CH₂O—CH₂CH₂—), ethylmethylaminoethyl (—CH₂CH₂N(CH₃)CH₂CH₂—), 1-ethoxy-2-(2-ethoxy-ethoxy)ethane (—CH₂CH₂O—CH₂CH₂—OCH₂CH₂—OCH₂CH₂—), and the like.

The term “alkaryl” refers to the groups -optionally substituted alkylene-optionally substituted aryl, where alkylene, substituted alkylene, aryl and substituted aryl are defined herein. Such alkaryl groups are exemplified by benzyl, phenethyl and the like.

The term “alkoxy” refers to the groups alkyl-O—, alkenyl-O—, cycloalkyl-O—, cycloalkenyl-O—, and alkynyl-O—, where alkyl, alkenyl, cycloalkyl, cycloalkenyl, and alkynyl are as defined herein. Alkoxy groups are alkyl-O— and include, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein. One substituted alkoxy group is substituted alkyl-O, and includes groups such as —OCH₂CH₂OCH₃, PEG groups such as —O(CH₂CH₂O)ₓCH₃, where x is an integer of 2-20, 2-10, and 2-5. Another substituted alkoxy group is —O—CH₂—(CH₂)_{y}—OH, where y is an integer of 1-10, or 14.

The term “alkylalkoxy” refers to the groups -alkylene-O-alkyl, alkylene-O-substituted alkyl, substituted alkylene-O-alkyl and substituted alkylene-O-substituted alkyl wherein alkyl, substituted alkyl, alkylene and substituted alkylene are as defined herein. Alkylalkoxy groups are alkylene-O-alkyl and include, by way of example, methylenemethoxy (—CH₂OCH₃), ethylenemethoxy (—CH₂CH₂OCH₃), n-propylene-iso-propoxy (—CH₂CH₂CH₂OCH(CH₃)₂), methylene-t-butoxy (—CH₂—O—C(CH₃)₃) and the like.

The term “alkylthioalkoxy” refers to the group -alkylene-O-alkyl, alkylene-O-substituted alkyl, substituted alkylene-O-alkyl and substituted alkylene-O-substituted alkyl wherein alkyl, substituted alkyl, alkylene and substituted alkylene are as defined herein. Other alkylthioalkoxy groups are alkylene-O-alkyl and include, by way of example, methylenethiomethoxy (—CH₂SCH₃), ethylenethiomethoxy (—CH₂CH₂SCH₃), n-propylene-iso-thiopropoxy (—CH₂CH₂CH₂SCH(CH₃)₂), methylene-t-thiobutoxy (—CH₂SC(CH₃)₃) and the like.

The term “alkenyl” refers to a monoradical of a branched or unbranched unsaturated hydrocarbon group having from 2 to 20 carbon atoms, or 2 to 10 carbon atoms, or 2 to 6 carbon atoms and having at least 1 and from 1-6 sites of vinyl unsaturation. Alkenyl groups include ethenyl (—CH═CH₂), n-propenyl (—CH₂CH═CH₂), iso-propenyl (—C(CH₃)═CH₂), and the like.

The term “substituted alkenyl” refers to an alkenyl group as defined above having from 1 to 5 substituents, 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “alkenylene” refers to a diradical of a branched or unbranched unsaturated hydrocarbon group having from 2 to 20 carbon atoms, 2 to 10 carbon atoms, or 2 to 6 carbon atoms and having at least 1 or from 1-6 sites of vinyl unsaturation. This term is exemplified by groups such as ethenylene (—CH═CH—), the propenylene isomers (e.g., —CH₂CH═CH— and —C(CH₃)═CH—) and the like.

The term “substituted alkenylene” refers to an alkenylene group as defined above having from 1 to 5 substituents, from 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl. Additionally, such substituted alkenylene groups include those where 2 substituents on the alkenylene group are fused to form one or more cycloalkyl, substituted cycloalkyl, cycloalkenyl, substituted cycloalkenyl, aryl, heterocyclic or heteroaryl groups fused to the alkenylene group.

The term “alkynyl” refers to a monoradical of an unsaturated hydrocarbon, having from 2 to 20 carbon atoms, or 2 to 10 carbon atoms, or 2 to 6 carbon atoms and having at least 1 or from 1-6 sites of acetylene (triple bond) unsaturation. Alkynyl groups include ethynyl, (—C≡CH), propargyl, (—C≡CCH₃), and the like.

The term “substituted alkynyl” refers to an alkynyl group as defined above having from 1 to 5 substituents, and 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “alkynylene” refers to a diradical of an unsaturated hydrocarbon having from 2 to 20 carbon atoms, 2 to 10 carbon atoms and 2 to 6 carbon atoms and having at least 1 and from 1-6 sites of acetylene (triple bond) unsaturation. Alkynylene groups include ethynylene (—C≡C—), propargylene (—CH₂—C≡C—) and the like.

The term “substituted alkynylene” refers to an alkynylene group as defined above having from 1 to 5 substituents, and 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “acyl” refers to the groups HC(O)—, allyl-C(O)—, substituted alkyl-C(O)—, cycloalkyl-C(O)—, substituted cycloalkyl-C(O)—, cycloalkenyl-C(O)—, substituted cycloalkenyl-C(O)—, aryl-C(O)—, heteroaryl-C(O)— and heterocyclic-C(O)— where allyl, substituted alkyl, cycloalkyl, substituted cycloalkyl, cycloalkenyl, substituted cycloalkenyl, aryl, heteroaryl and heterocyclic are as defined herein.

The term “acylamino” or “aminocarbonyl” refers to the group —C(O)NRR where each R is independently hydrogen, allyl, substituted alkyl, aryl, heteroaryl, heterocyclic or where both R groups are joined to form a heterocyclic group (e.g., morpholino) wherein alkyl, substituted allyl, aryl, heteroaryl and heterocyclic are as defined herein.

The term “aminoacyl” refers to the group —NRC(O)R where each R is independently hydrogen, alkyl, substituted alkyl, aryl, heteroaryl, or heterocyclic wherein alkyl, substituted alkyl, aryl, heteroaryl and heterocyclic are as defined herein.

The term “aminoacyloxy” or “alkoxycarbonylamino” refers to the group —NRC(O)OR where each R is independently hydrogen, alkyl, substituted alkyl, aryl, heteroaryl, or heterocyclic wherein alkyl, substituted alkyl, aryl, heteroaryl and heterocyclic are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclic-C(O)O— wherein alkyl, substituted alkyl, cycloalkyl, substituted cycloalkyl, aryl, heteroaryl, and heterocyclic are as defined herein.

The term “aryl” refers to an unsaturated aromatic carbocyclic group of from 6 to 20 carbon atoms having a single ring (e.g., phenyl) or multiple condensed (fused) rings (e.g., naphthyl or anthryl). Aryls include phenyl, naphthyl and the like.

Unless otherwise constrained by the definition for the aryl substituent, such aryl groups can optionally be substituted with from 1 to 5 substituents, 1 to 3 substituents, selected from the group consisting of acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl. Aryl substituents include alkyl, alkoxy, halo, cyano, nitro, trihalomethyl, and thioalkoxy.

The term “aryloxy” refers to the group aryl-O— wherein the aryl group is as defined above including optionally substituted aryl groups as also defined above.

The term “arylene” refers to the diradical derived from aryl (including substituted aryl) as defined above and is exemplified by 1,2-phenylene, 1,3-phenylene, 1,4-phenylene, 1,2-naphthylene and the like.

The term “amino” refers to the group —NH₂.

The term “substituted amino refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic provided that both R's are not hydrogen.

The term “carboxyalkyl” or “alkoxycarbonyl” refers to the groups “—C(O)O-alkyl”, “—C(O)O-substituted alkyl”, “—C(O)O-cycloalkyl”, “—C(O)O-substituted cycloalkyl”, “—C(O)O-alkenyl”, “—C(O)O-substituted alkenyl”, “—C(O)O-alkynyl” and “—C(O)O-substituted alkynyl” where alkyl, substituted alkyl, cycloalkyl, substituted cycloalkyl, alkenyl, substituted alkenyl, alkynyl and substituted alkynyl are as defined herein.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The term “cycloalkylene” refers to the diradical derived from cycloalkyl as defined above and is exemplified by 1,1-cyclopropylene, 1,2-cyclobutylene, 1,4-cyclohexylene and the like.

The term “substituted cycloalkyl” refers to cycloalkyl groups having from 1 to 5 substituents, and 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “substituted cycloalkylene” refers to the diradical derived from substituted cycloalkyl as defined above.

The term “cycloalkenyl” refers to cyclic alkenyl groups of from 4 to 20 carbon atoms having a single cyclic ring and at least one point of internal unsaturation. Examples of suitable cycloalkenyl groups include, for instance, cyclobut-2-enyl, cyclopent-3-enyl, cyclooct-3-enyl and the like.

The term “cycloalkenylene” refers to the diradical derived from cycloalkenyl as defined above and is exemplified by 1,2-cyclobut-1-enylene, 1,4-cyclohex-2-enylene and the like.

The term “substituted cycloalkenyl” refers to cycloalkenyl groups having from 1 to 5 substituents, 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “substituted cycloalkenylene” refers to the diradical derived from substituted cycloalkenyl as defined above.

The term “halo” or “halogen” refers to fluoro, chloro, bromo and iodo.

The term “heteroaryl” refers to an aromatic group comprising 1 to 15 carbon atoms and 1 to 4 heteroatoms selected from oxygen, nitrogen and sulfur within at least one ring (if there is more than one ring).

Unless otherwise constrained by the definition for the heteroaryl substituent, such heteroaryl groups can be optionally substituted with 1 to 5 substituents, 1 to 3 substituents, selected from the group consisting of acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl. Aryl substituents include alkyl, alkoxy, halo, cyano, nitro, trihalomethyl, and thioalkoxy. Such heteroaryl groups can have a single ring (e.g., pyridyl or furyl) or multiple condensed rings (e.g., indolizinyl or benzothienyl). Heteroaryls include pyridyl, pyrrolyl and furyl.

The term “heteroaryloxy” refers to the group heteroaryl-O—.

The term “heteroarylene” refers to the diradical group derived from heteroaryl (including substituted heteroaryl), as defined above, and is exemplified by the groups 2,6-pyridylene, 2,4-pyridiylene, 1,2-quinolinylene, 1,8-quinolinylene, 1,4-benzofuranylene, 2,5-pyridinylene, 2,5-indolenyl and the like.

The term “heterocycle” or “heterocyclic” refers to a monoradical saturated or unsaturated group having a single ring or multiple condensed rings, having from 1 to 40 carbon atoms and from 1 to 10 hetero atoms, 1 to 4 heteroatoms, selected from nitrogen, sulfur, phosphorus, and/or oxygen within the ring.

Unless otherwise constrained by the definition for the heterocyclic substituent, such heterocyclic groups can be optionally substituted with 1 to 5, 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl. Such heterocyclic groups can have a single ring or multiple condensed rings. Heterocyclics include morpholino, piperidinyl, and the like.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles.

The term “heterocyclooxy” refers to the group heterocyclic-O—.

The term “thioheterocyclooxy” refers to the group heterocyclic-S—.

The term “heterocyclene” refers to the diradical group formed from a heterocycle, as defined herein, and is exemplified by the groups 2,6-morpholino, 2,5-morpholino and the like.

The term “oxyacylamino” or “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

The term “spiro-attached cycloalkyl group” refers to a cycloalkyl group attached to another ring via one carbon atom common to both rings.

The term “thiol” refers to the group —SH.

The term “thioalkoxy” refers to the group —S-alkyl.

The term “substituted thioalkoxy” refers to the group —S-substituted alkyl.

The term “thioaryloxy” refers to the group aryl-S— wherein the aryl group is as defined above including optionally substituted aryl groups also defined above.

The term “thioheteroaryloxy” refers to the group heteroaryl-S— wherein the heteroaryl group is as defined above including optionally substituted aryl groups as also defined above.

The term “carboxyamides” include primary carboxyamides (CONH₂), secondary carboxyamides (CONHR′) and tertiary carboxyamides (CONR′R″), where R′ and R″ are the same or different substituent groups chosen from alkyl, alkenyl, alkynyl, alkoxy, aryl, a heterocyclic group, a functional group as defined herein, and the like, which themselves may be substituted or unsubstituted.

“Carboxyamidealkyl” means a carboxyamide as defined above attached to an optionally substituted alkylene group as defined above.

The term “saccharide” includes oxidized, reduced or substituted saccharides, including hexoses such as D-glucose, D-mannose or D-galactose; pentoses such as D-ribose or D-arabinose; ketoses such as D-ribulose or D-fructose; disaccharides such as sucrose, lactose, or maltose; derivatives such as acetals, amines, and phosphorylated sugars; oligosaccharides; as well as open chain forms of sugars, and the like. Examples of amine-derivatized sugars are galactosamine, glucosamine, and sialic acid.

As to any of the above groups that contain one or more substituents, it is understood, of course, that such groups do not contain any substitution or substitution patterns which are sterically impractical and/or synthetically non-feasible. In addition, the compounds of this application include all stereochemical isomers arising from the substitution of these compounds.

### Zinc

Zinc is a co-factor in a variety of cellular processes including DNA synthesis, behavioral responses, reproduction, bone formation, growth and wound healing. Zinc is a component of insulin and it plays a role in the efficacy of most of the functions of your body. Zinc is necessary for the free-radical quenching activity of superoxide dismutase (SOD), an antioxidant enzyme which breaks down the free-radical superoxide to form hydrogen peroxide. The abundance of loosely-bound or free intracellular zinc can impact on cellular metabolism, survival and growth. Zinc may aid in the prevention and treatment of cancer. The methods of the present application provide for a method of treating animal subjects suffering from a disease with abnormal proliferation or abnormal cell death, which involves the administration of a combination of an effective amount of metal-containing texaphyrin (and in one embodiment a lanthanide-containing texaphyrin) of Formula I or Formula II and an effective amount of a zinc (II) reagent. Examples of zinc (II) reagents that can be used in the methods of the present application include, but are not limited, to zinc acetate, zinc chloride, zinc citrate, zinc lactate zinc gluconate, L-carnosine salt, zinc fetuin, zinc sulfate, zinc bacitracin, zinc seleno-bacitracin, chelated zinc, and zinc ionophores such as zinc 1-hydroxypyridine-2-thiol.

### Cellular Mechanisms

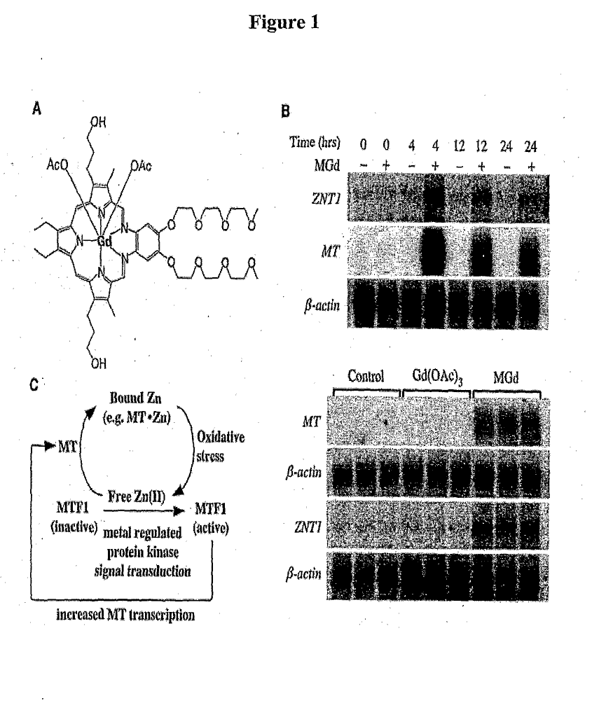

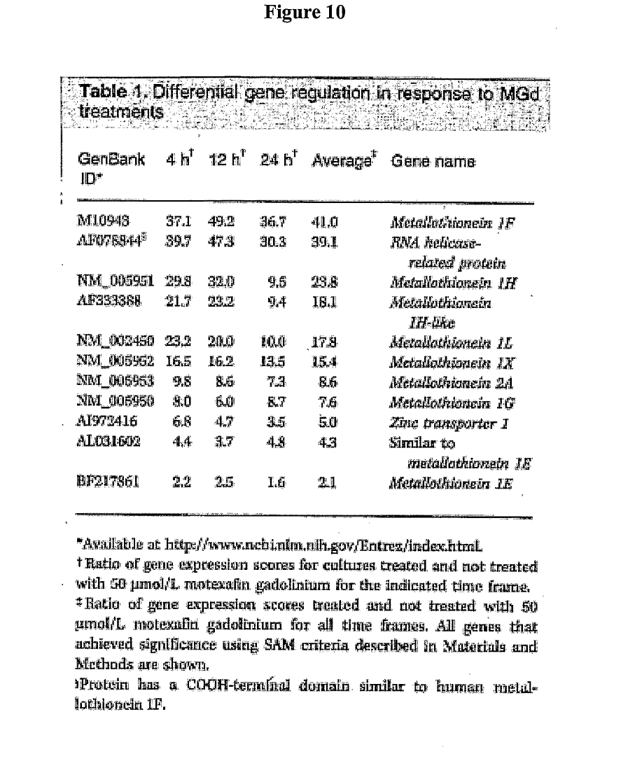

A549 lung cancer cell cultures treated with MGd at multiple exposure times showed changes in mRNA levels. These cells showed a highly specific response that consisted of a strong and sustained induction of metallothionein and zinc transporter 1 (ZnT1) transcripts (see FIG. 10). Metallothioneins possess multiple cysteine-rich sites that can bind metal cations. They are expressed constitutively, and may be further induced by toxic metal cations such as Cd (II), or by non-toxic cations such as Zn (II). ZnT1 is a plasma membrane-bound protein that transports zinc to the outside of the cell. The transcription of metallothionein genes and ZnT1 is induced by the metal response element-binding transcription factor-1 (MTF-1), a metal-dependent transactivator that binds to metal response elements (MREs) located in the promoters of metallothionein and other genes (see FIG. 1C).

Zinc metallothioneins are believed to be a site of intracellular zinc storage and transport. As shown in FIG. 1C, oxidation of zinc metallothionein by hydrogen peroxide leads to the formation of thionein and the release of zinc. The disulfide bond in thionein must be reduced by thioredoxin reductase, in order to re-coordinate to zinc. The released zinc can bind and activate MTF-1, leading, in turn, to up-regulation of thionein expression. The binding of zinc by the newly transcribed thioneins provides a negative feedback mechanism for metallothionein expression.

MGd is a redox active agent that has been shown to redox cycle and form reactive oxygen species in cells. Other redox cycling agents have been shown to induce metallothionein expression. Interestingly, recent data suggests that MGd oxidizes vicinal thiols such as dithiothreitol and can inhibit thioredoxin reductase. Without being limited to any theory, it is therefore possible that the generation of reactive oxygen species, the direct oxidation of zinc metallothionein by the complex, or thioredoxin reductase inhibition could be responsible for the observed metallothionein induction. MGd treatment affects a subset of the genes reported to be induced or repressed by oxidative stress in cultured mammalian cells.

In one aspect, subjects are monitored prior to and/or after treatment with a metal-containing texaphyrin (and in one embodiment a lanthanide-containing texaphyrin) in order to predict treatment efficacy, modulate the administration of treatment or consider other alternatives. In one embodiment, the administration of a metal-containing texaphyrin (and in one embodiment a lanthanide-containing texaphyrin) will be used for treating tumors, atheroma and other neoplastic tissue as well as other conditions that are responsive to the induction of targeted oxidative stress and/or changes in cellular zinc levels. In one embodiment, the administration of an effective amount of a metal-containing texaphyrin (and in one embodiment a lanthanide-containing texaphyrin) induces metallothionein and/or ZnT1 transcripts in targeted cells. Methods for measuring expression, induction and or activation of metallothionein and ZnT1 are known in the art, including those disclosed herein. In another embodiment, the induction of metallothionein and/or ZnT1 is indicative of treatment efficacy. In yet another embodiment, the administration of the metal-containing texaphyrin (and in one embodiment a lanthanide-containing texaphyrin) is modulated according to the induction of metallothionein and/or ZnT1 in targeted cells. In one embodiment, the administration of an effective amount a metal-containing texaphyrin (and in one embodiment a lanthanide-containing texaphyrin) induces MTF-1 in targeted cells. Methods for measuring expression, induction and or activation MTF-1 are known in the art, including those disclosed herein. In another embodiment, the induction of MTF-1 is indicative of treatment efficacy. In yet another embodiment, the administration of the metal-containing texaphyrin (and in one embodiment a lanthanide-containing texaphyrin) is modulated according to the induction of MTF-1 in targeted cells.

In another aspect, subjects are monitored prior of after treatment with compounds that induce oxidative stress and/or changes in cellular zinc levels on target cells in order to predict treatment efficacy, modulate the administration of treatment or consider other alternatives. Methods for measuring oxidative stress and/or changes in cellular zinc levels are well known in the art, including those disclosed herein. In one embodiment, the administration of such compounds induces metallothionein and/or ZnT1 transcripts in targeted cells. In another embodiment, the induction of metallothionein and/or ZnT1 is indicative of treatment efficacy. In yet another embodiment, the administration of the compounds is modulated according to the induction of metallothionein and/or ZnT1 in targeted cells. In one embodiment, the administration of such compounds induces MTF-1 in targeted cells. In another embodiment, the induction of MTF-1 is indicative of treatment efficacy. In yet another embodiment the administration of the compounds is modulated according to the induction of MTF-1 in targeted cells.

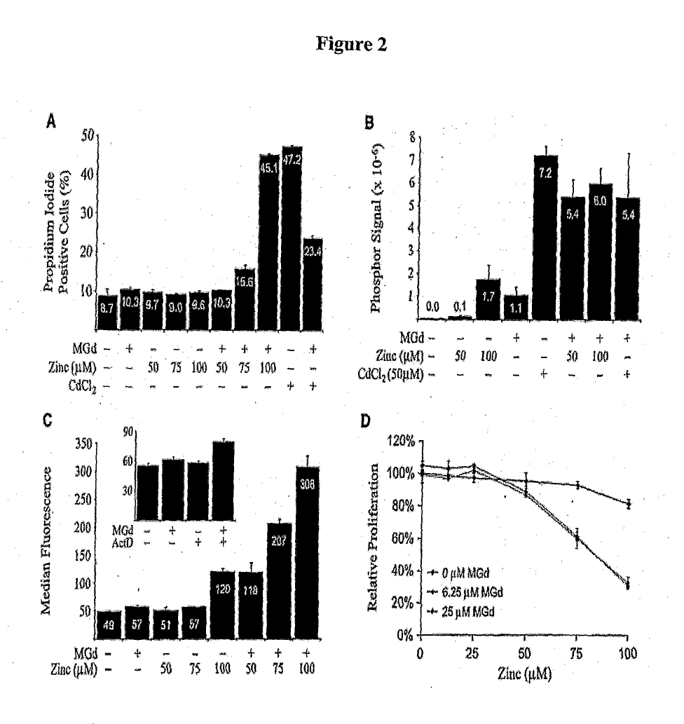

Treatment with MGd attenuated the cytotoxicity of CdCl₂ and potentiated that of Zn(OAc)₂ in A549 cells (FIG. 2A). Metallothionein transcript levels were raised by treatment with MGd, zinc, cadmium, or combinations of these species (FIG. 2B). Intracellular levels of free zinc were examined using the ion-specific probe, FluoZin-3 (Kd=15 nM), and observed significantly (at least 2.4-fold) increased cellular fluorescence signals following co-incubation with MGd and 50-100 μM zinc for 4 hours (FIG. 2C). Synergistic increases in intracellular free zinc levels in response to co-incubation with MGd and zinc acetate could explain the cellular toxicity observed. Furthermore, the 1.5-fold increase in cellular fluorescence signal observed by co-incubating A549 cells with MGd and actinomycin D in zinc-free medium (inset, FIG. 2C) suggests that MGd-treatment can mobilize bound intracellular zinc. This mobilization is normally quenched by cellular gene expression responses, most likely those of metallothionein gene family members and ZnT1, since MGd treatment in the absence of actinomycin D only led to marginal increases in cellular fluorescence in both serum and serum-free media (1.2 and 1.1-fold, respectively, in A549).

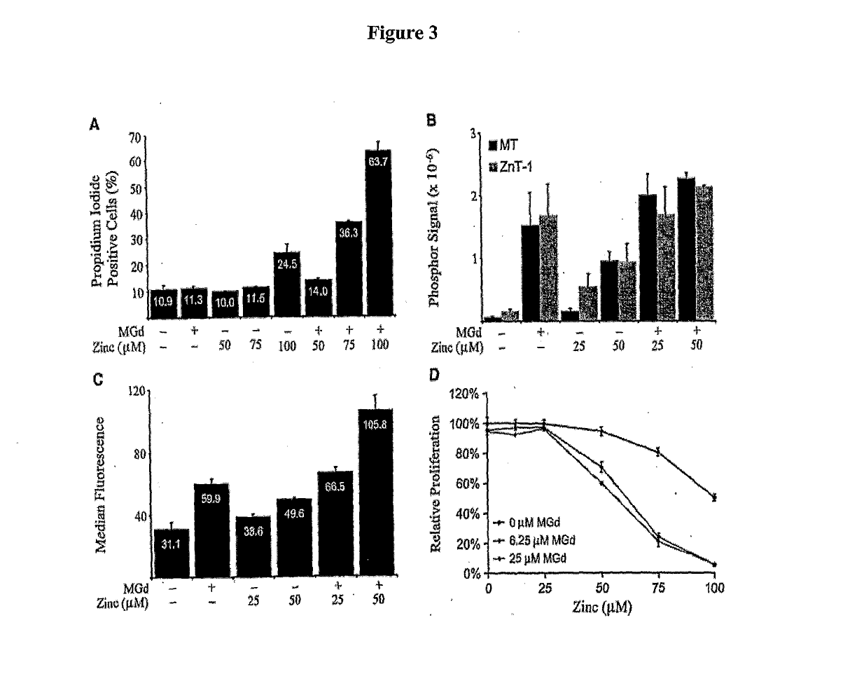

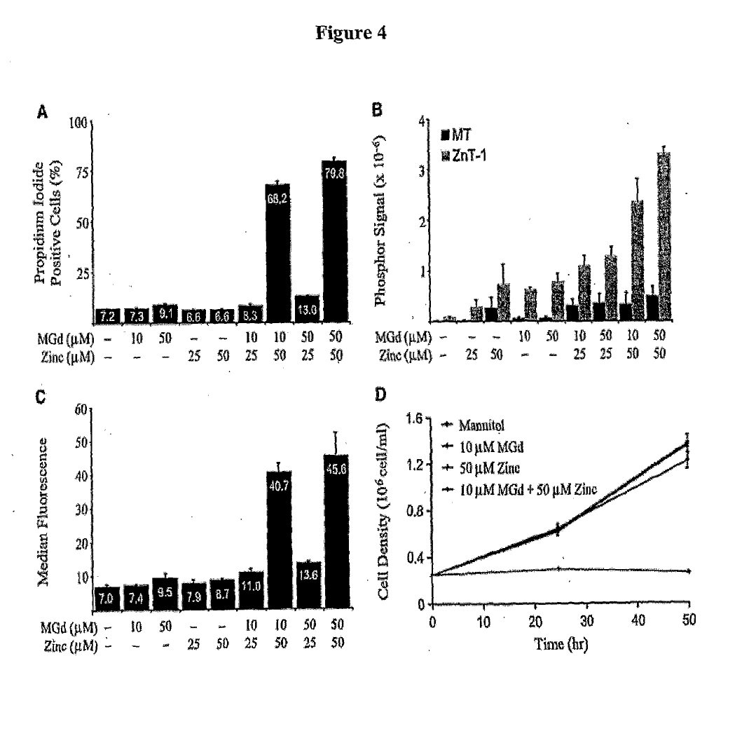

Similar effects were observed in PC3 prostate cancer and Ramos B-cell lymphoma cell lines. Combined treatment with MGd and zinc led to increased cell death after 48 hours in PC3 cultures (FIG. 3A) and after 24 hours in Ramos cultures (FIG. 4A). Expression of metallothionein family members and ZnT1 were increased by MGd in both lines (FIGS. 3B and 4B). Larger changes in FluoZin-3 fluorescence (approximately 2-fold) were observed in PC3 cultures treated with MGd alone than in the A549 or Ramos lines (FIGS. 2C, 3C, and 4C). The difference could result from the lesser induction of gene expression of metallothionein gene family members or ZnT1 in this line.

In another aspect, metal-containing texaphyrin (and in one embodiment a lanthanide-containing texaphyrin)s and/or zinc (II) reagents will be used for treating tumors, atheromas and other neoplastic tissue as well as other conditions that are responsive to the induction of targeted oxidative stress and/or changes in cellular zinc levels. In one embodiment, the administration of an effective amount a metal-containing texaphyrin (and in one embodiment a lanthanide-containing texaphyrin) and/or an effective amount of a zinc (II) reagent will induce cell death in target cells.

Yet another aspect is to monitor alterations in zinc homeostasis in plasma and in target cells prior to and/or after treatment as a predictor for treatment efficacy. In one embodiment, an increase in the levels of intracellular zinc in plasma and in target cells will be used as a predictor of treatment efficacy. In another embodiment, the levels of intracellular zinc in plasma and in target cells will be measured prior to treatment with an effective amount a metal-containing texaphyrin (and in one embodiment a lanthanide-containing texaphyrin) and/or an effective amount of a zinc (II) reagent. Methods for measuring intracellular zinc levels are known in the art, including those disclosed herein. In yet another embodiment, the administration of the metal-containing texaphyrin (and in one embodiment a lanthanide-containing texaphyrin) and the zinc (II) reagent is modulated according to the levels of intracellular zinc in plasma and on target cells prior to and/or after treatment. In another embodiment, the levels of intracellular zinc in the target cells will be measured prior to treatment with compounds that induce and increase intracellular zinc levels. In another embodiment, the administration of such compounds is modulated according to the levels of intracellular zinc in plasma and on target cells prior to and/or after treatment.

Another aspect is to monitor different genes involved in response to treatment with metal-containing texaphyrin (and in one embodiment a lanthanide-containing texaphyrin)s and/or zinc prior to and/or after treatment as predictors for treatment efficacy. In one embodiment, the administration of an effective amount a metal-containing texaphyrin (and in one embodiment a lanthanide-containing texaphyrin) and/or an effective amount of a zinc (II) reagent induces metallothionein and/or ZnT1 transcripts in targeted cells. In one embodiment, the administration of other compounds induces metallothionein and/or ZnT1 transcripts in targeted cells. Methods for measuring induction, expression and/or activation of metallothionein and ZnT1 are known in the art, including those disclosed herein. In another embodiment, the induction of metallothionein and/or ZnT1 is indicative of treatment efficacy. In another embodiment, the administration of the metal-containing texaphyrin (and in one embodiment a lanthanide-containing texaphyrin) and the zinc (II) reagent is modulated according to the induction of metallothionein and/or ZnT1 in targeted cells. In yet another embodiment, the administration of compounds that induce metallothionein and/or ZnT1 on target cells will be modulated according to the induction of metallothionein and/or ZnT1 in targeted cells.

In one embodiment, the administration of an effective amount a metal-containing texaphyrin (and in one embodiment a lanthanide-containing texaphyrin) and/or an effective amount of a zinc (II) reagent induces MTF-1 in targeted cells. In another embodiment, the induction of MTF-1 is indicative of treatment efficacy. In yet another embodiment, the administration of the metal-containing texaphyrin (and in one embodiment a lanthanide-containing texaphyrin) and/or the zinc (II) reagent is modulated according to the induction of MTF-1 in targeted cells.

In one embodiment, the administration of an effective amount of a metal-containing texaphyrin (and in one embodiment a lanthanide-containing texaphyrin) and/or an effective amount of a zinc (II) reagent induces the oxidation of vicinal thiols such as dithiothreitol. Methods for measuring the oxidation of vicinal thiols are known in the art, including those disclosed herein. In another embodiment, oxidation of vicinal thiols is indicative of treatment efficacy. In yet another embodiment, the administration of the metal-containing texaphyrin (and in one embodiment a lanthanide-containing texaphyrin) and the zinc (II) reagent is modulated according to the oxidation of vicinal thiols in targeted cells. In one embodiment, the administration of an effective amount of a metal-containing texaphyrin (and in one embodiment a lanthanide-containing texaphyrin) and/or an effective amount of a zinc (II) reagent inhibits thioredoxin reductase. In another embodiment, the inhibition of thioredoxin reductase is indicative of treatment efficacy. Methods for measuring the inhibition of thioredoxin reductase are known in the art, including those disclosed herein. In yet another embodiment, the administration of the metal-containing texaphyrin (and in one embodiment a lanthanide-containing texaphyrin) and/or the zinc (II) reagent is modulated according to the inhibition of thioredoxin reductase.

In one embodiment, the administration of compounds that act through the same cellular mechanism as a metal-containing texaphyrin (and in one embodiment a lanthanide-containing texaphyrin) and a zinc (II) reagent will be modulated according to the oxidation of vicinal thiols such as dithiothreitol, and the inhibition of thioredoxin reductase.

Another aspect is to treat tumors, atheromas and other neoplastic tissue as well as other conditions that are responsive to the induction of targeted oxidative stress and/or changes in cellular zinc levels with an effective amount of a metal-containing texaphyrin (and in one embodiment a lanthanide-containing texaphyrin) and/or an effective amount of a zinc (II) reagent in combination with compounds that regulate genes involved in the response to treatment with metal-containing texaphyrin (and in one embodiment a lanthanide-containing texaphyrin)s and/or zinc. The compounds can, for example, activate or inhibit genes involved in the response to treatment with metal-containing texaphyrin (and in one embodiment a lanthanide-containing texaphyrin)s and/or zinc. In one embodiment, an effective amount of metal-containing texaphyrin (and in one embodiment a lanthanide-containing texaphyrin) and/or an effective amount of a zinc (II) reagent is administered in combination with an effective amount of a compound that regulates metallothionein and/or ZnT1 transcripts in target cells. In another embodiment, an effective amount of metal-containing texaphyrin (and in one embodiment a lanthanide-containing texaphyrin) and/or an effective amount of a zinc (II) reagent is administered in combination with an effective amount of a compound that regulates MTF-1 in target cells. In another embodiment, an effective amount of metal-containing texaphyrin (and in one embodiment a lanthanide-containing texaphyrin) and/or an effective amount of a zinc (II) reagent is administered in combination with an effective amount of a compound that regulates oxidation of vicinal thiols such as dithiothreitol in target cells. In another embodiment, an effective amount of metal-containing texaphyrin (and in one embodiment a lanthanide-containing texaphyrin) and/or an effective amount of a zinc (II) reagent is administered in combination with an effective amount of a compound that regulates the inhibition of thioredoxin reductase.

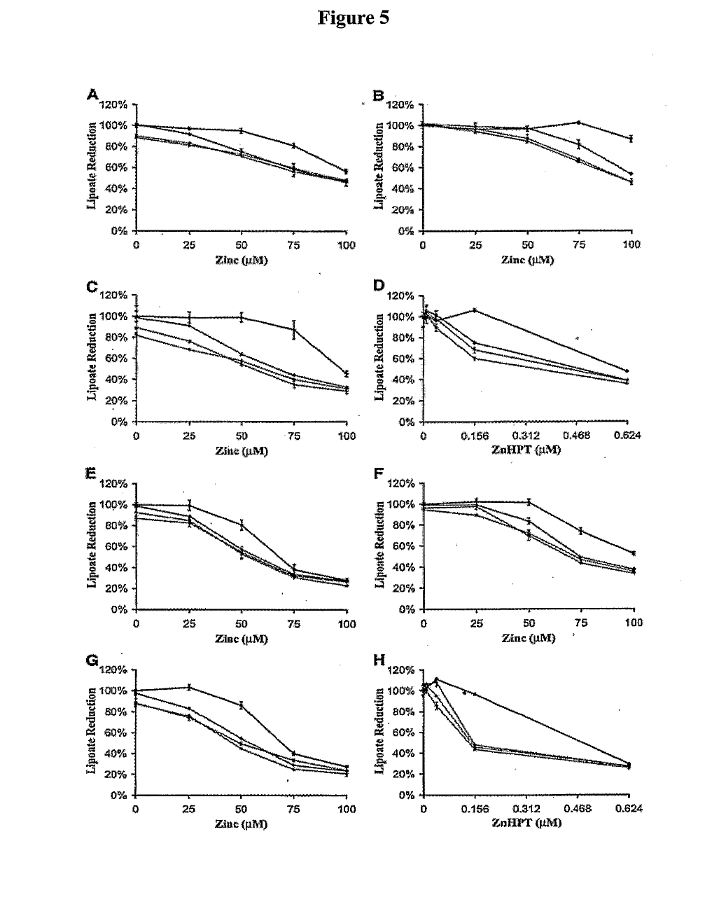

Increased intracellular zinc levels would also explain the effect of MGd and zinc on lipoate reduction (FIG. 5). Lipoate is reduced by thioredoxin reductase in mammalian cells, accounting for approximately two-thirds of this activity in A549 cultures, with the remainder due to glutaredoxin or other enzymes. Inhibition of thioredoxin reductase by zinc in cell extracts was reported previously. The data in FIG. 5 show that zinc inhibition of thioredoxin reductase occurs in intact cells. MGd alone had an effect on the rate of lipoate reduction under experimental conditions proved herein after 2 hours of treatment (y-axis, FIG. 5A). Moreover, inhibition of lipoate reduction by zinc was potentiated by MGd. The effect of MGd was dose-dependent, and saturated above a concentration of approximately 5 μM. The inhibition of lipoate reduction was less pronounced after 4 hours of incubation (FIG. 5B). However, pretreatment with either actinomycin D (FIG. 5C) or cycloheximide (data not shown) restored the inhibitory effect of both MGd and zinc. This is reminiscent of the effect of actinomycin D on the fluorescence measurements described above (FIG. 2C), and is consistent with compensatory cellular RNA and protein expression in response to MGd. Lipoate reduction was also inhibited in cells treated with a zinc ionophore (FIG. 5D), demonstrating that the effect of MGd was independent of the mode of zinc uptake. Lipoate reduction was similarly inhibited by zinc and MGd in PC3 and Ramos cultures (FIGS. 5E-H, and data not shown). In one embodiment, the administration of an effective amount a metal-containing texaphyrin (and in one embodiment a lanthanide-containing texaphyrin) and/or an effective amount of a zinc (II) reagent induces the inhibition of lipoate reduction. In another embodiment, inhibition of lipoate reduction is indicative of treatment efficacy. In yet another embodiment, the administration of the metal-containing texaphyrin (and in one embodiment a lanthanide-containing texaphyrin) and/or the zinc (II) reagent is modulated according to the inhibition of lipoate reduction in targeted cells. In another embodiment, an effective amount of metal-containing texaphyrin (and in one embodiment a lanthanide-containing texaphyrin) and/or an effective amount of a zinc (II) reagent is administered in combination with an effective amount of a compound that regulates lipoate reduction.

Thioredoxin reductase is a component of the cellular anti-oxidant system, and is involved in a variety of other processes including apoptotic signaling and DNA synthesis. It has recently been highlighted as an attractive target for anticancer agent activity. Combined treatment with MGd and zinc inhibited cell proliferation (FIGS. 2D, 3D, 4D), and led to cell death (FIGS. 2A, 3A, and 4A). Similar observations were made using the HF-1 and DHL-4 B-lymphoma cell lines (data not shown). Not intended to be limited to any mechanism of action, Thioredoxin reductase inhibition could contribute to the observed effects. In one embodiment, the administration of an effective amount a metal-containing texaphyrin (and in one embodiment a lanthanide-containing texaphyrin) and/or an effective amount of a zinc (II) reagent inhibits thioredoxin reductase. In another embodiment, the inhibition of thioredoxin reductase is indicative of treatment efficacy. In yet another embodiment, the administration of the metal-containing texaphyrin (and in one embodiment a lanthanide-containing texaphyrin) and the zinc (II) reagent is modulated according to the inhibition of thioredoxin reductase. In another embodiment, an effective amount of metal-containing texaphyrin (and in one embodiment a lanthanide-containing texaphyrin) and/or an effective amount of a zinc (II) reagent is administered in combination with an effective amount of a compound that regulates the inhibition of thioredoxin reductase.

In one embodiment, the administration of compounds that act through the same cellular mechanism as a metal-containing texaphyrin (and in one embodiment a lanthanide-containing texaphyrin) and a zinc (ID reagent will be modulated according to the inhibition of lipoate reduction, and the inhibition of thioredoxin reductase.

Many other proteins require zinc for activity, and may therefore be affected by changes in the intracellular concentration of available zinc. The observation of a sustained induction of ZnT1 and metallothionein transcripts suggests a corresponding increase in the intracellular availability of zinc during drug treatment. MGd could therefore modulate a variety of downstream processes by mobilizing zinc. The importance of this would likely depend on the particular system under study, but would be most likely to occur in tumors, where the drug appears to localize selectively. In one embodiment, the administration of an effective amount a metal-containing texaphyrin (and in one embodiment a lanthanide-containing texaphyrin) and/or an effective amount of a zinc (II) reagent increases intracellular levels of zinc in tumor cells. In another embodiment, the increased levels in intracellular zinc in tumor cells are indicative of treatment efficacy. In yet another embodiment, the administration of the metal-containing texaphyrin (and in one embodiment a lanthanide-containing texaphyrin) and the zinc (II) reagent is modulated according to the increased levels in intracellular zinc in tumor cells. In one embodiment, the levels of zinc in tumor cells will be used to determine treatment efficacy prior to treatment with the metal-containing texaphyrin (and in one embodiment a lanthanide-containing texaphyrin) and the zinc (II) reagent. In another embodiment the dosage of the metal-containing texaphyrin (and in one embodiment a lanthanide-containing texaphyrin) and the zinc (II) reagent will be determined according to the intracellular zinc levels in tumor cells prior to treatment.

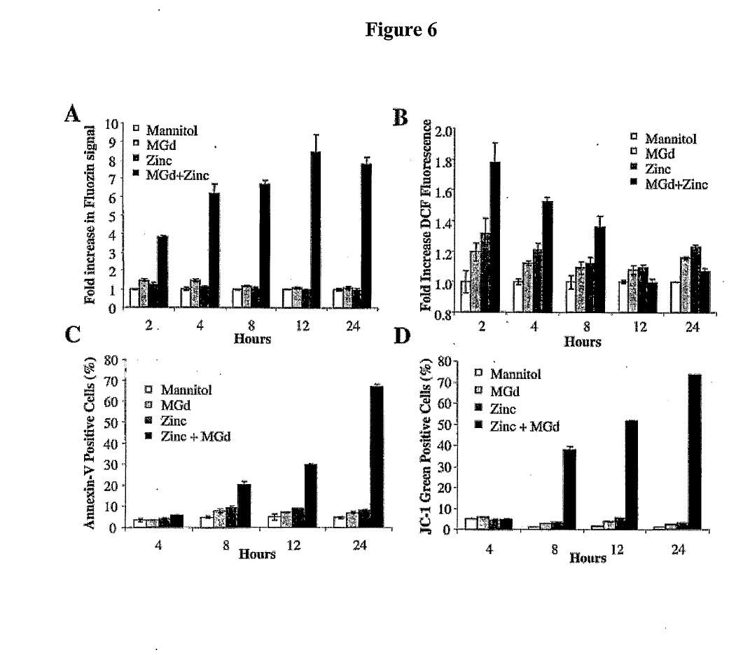

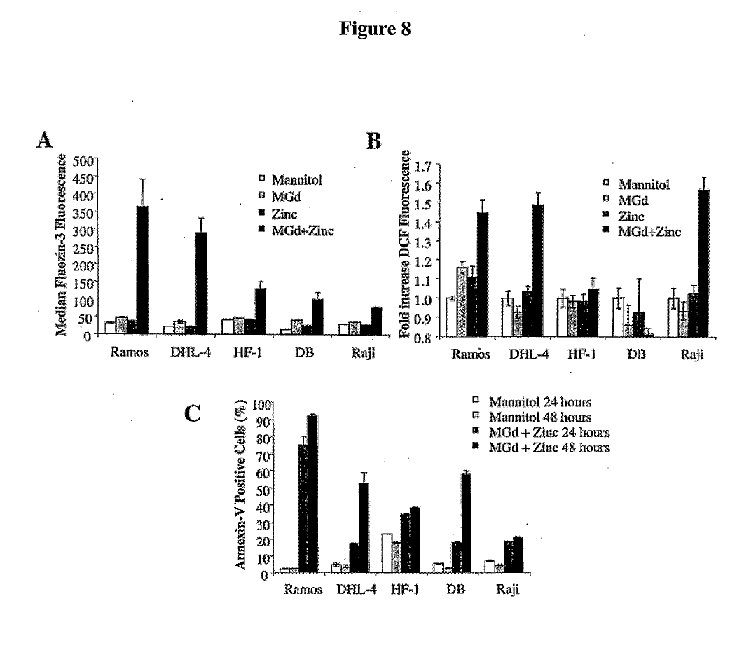

MGd represents a class of compounds capable of altering the expression of MTF-1 responsive genes and altering zinc homeostasis in cancer cells. As shown above the cellular activity of 10 μM MGd was enhanced in the presence of 25 and 50 μM exogenous zinc, these zinc concentrations were used in the following experiments. They are also relevant given that (i) standard tissue culture conditions are deficient in zinc, having an estimated three to six-fold lower concentration (ca. 4 μM) as compared to normal human plasma samples and (ii) interstitial fluid zinc concentrations can vary greatly in vivo. Within two hours of MGd and/or zinc treatment, Ramos cells showed significant increases in intracellular free zinc (FIG. 6A). These levels continued to rise for at least 12 hours and remained high, at least in the group co-treated with MGd and zinc. This could represent a catastrophic loss of zinc homeostasis in these cells due to an overwhelming of cellular stress responses. Four other B-cell lines treated with MGd and zinc displayed increased intracellular free zinc levels after 4 hours (FIG. 8A), albeit to variable degrees.

Substantial increases in levels of reactive oxygen species (ROS) in Ramos cells within two hours of treatment with 10 μM MGd and/or 50 μM zinc was also observed (FIG. 6B). However, in contrast to the intracellular free zinc levels described above, ROS decreased over the course of the 24 hour treatment.

In keeping with their differences in intracellular free zinc, four other B-cell lines displayed variable degrees of oxidative stress after 4 hours of co-treatment with MGd and zinc (FIG. 8B). Treatment of Ramos cultures with hydrogen peroxide also led to transient increases in oxidative stress and sustained increases in intracellular free zinc. This is consistent with observations that zinc can induce oxidative stress in cultured mammalian cells, and, conversely, that thiol oxidation can mobilize zinc. In one embodiment, the administration of an effective amount of a metal-containing texaphyrin (and in one embodiment a lanthanide-containing texaphyrin) and/or an effective amount of a zinc (II) reagent increases oxidative stress in tumor cells. In another embodiment, the increase in oxidative stress in tumor cells is indicative of treatment efficacy. In yet another embodiment, the administration of the metal-containing texaphyrin (and in one embodiment a lanthanide-containing texaphyrin) and the zinc (II) reagent is modulated according to the increased levels in oxidative stress in tumor cells. In another embodiment, the administration of compounds that act through the same cellular mechanism as a metal-containing texaphyrin (and in one embodiment a lanthanide-containing texaphyrin) and a zinc (II) reagent will be modulated according to oxidative stress in tumor cells.

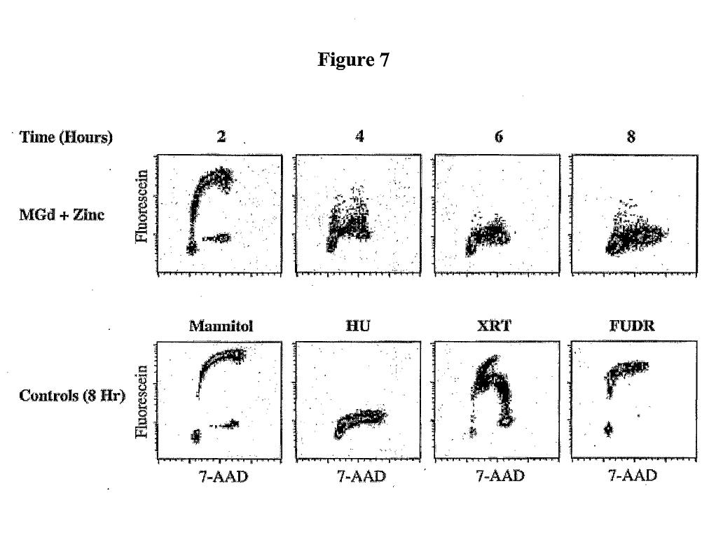

In addition, intracellular free zinc and oxidative stress were related to cellular growth rate. A large reduction in the number of Ramos cells actively synthesizing DNA in S-phase after treatment with MGd and 50 μM zinc by four hours was observed (FIG. 7). Other cell lines tested also displayed decreased DNA synthesis under these conditions. Treatment with 50 to 100 μM zinc inhibited the proliferation of four additional B-cell lines, an acute myelogenous leukemia line (K562), and a T-cell lymphoma line (Jurkat), but not in an acute promyelocytic leukemia line (HL60). In all lines except HL60, MGd co-treatment potentiated the inhibition by zinc. The effect of MGd and zinc differed from that of 5-fluoro-2′-deoxyuridine or ionizing radiation, both of which permitted BrdU incorporation into DNA, and changed cell cycle distribution with accumulation of cells in G1/S and G2/M, respectively. It also differed from hydroxyurea, which inhibited BrdU incorporation but allowed passage through G2M. Not intending to be limited by one mechanism of action, this suggests that increased intracellular free zinc inhibits proliferation at multiple checkpoints. In one embodiment, the administration of an effective amount of a metal-containing texaphyrin (and in one embodiment a lanthanide-containing texaphyrin) and/or an effective amount of a zinc (II) reagent decreases cellular growth in tumor cells. In another embodiment, the level of intracellular zinc and/or oxidative stress will be monitored prior to and after treatment with the metal-containing texaphyrin (and in one embodiment a lanthanide-containing texaphyrin) and/or the zinc (II) reagent, or compounds acting through the same mechanisms. Methods for measuring intracellular zinc and/or oxidative stress are known in the art, including those described herein. The levels of intracellular zinc and/or oxidative stress in tumor cells can then be used as predictors for cellular growth inhibition on tumor cells. In yet another embodiment, the administration of the metal-containing texaphyrin (and in one embodiment a lanthanide-containing texaphyrin) and/or the zinc (II) reagent, or other compounds acting through the same mechanisms, is modulated according to the increased levels in intracellular zinc and/or oxidative stress in tumor cells in order to decrease cellular growth.

The increased oxidative stress and intracellular free zinc levels induced by co-treatments with MGd and zinc preceded mitochondrial dysfunction and early events of apoptosis and thus were not a consequence of them. Furthermore, increased intracellular free zinc and oxidative stress roughly correlate with cell death, with Ramos the most sensitive line, followed by DHL-4 and the others (FIG. 8C). However, intracellular free zinc levels appeared to be better predictors of proliferative and apoptotic response. K562, HL60, and Jurkat lines did not exhibit changes in oxidative stress, intracellular free zinc, or apoptosis under these conditions. In one embodiment, the administration of an effective amount a metal-containing texaphyrin (and in one embodiment a lanthanide-containing texaphyrin) and/or an effective amount of a zinc (II) reagent induces proliferative and apoptosis response in tumor cells. In another embodiment, an increase in the levels of intracellular zinc in tumor cells after treatment with a metal-containing texaphyrin (and in one embodiment a lanthanide-containing texaphyrin) and/or a zinc (II) reagent, or other compounds acting through the same mechanisms, is indicative of proliferative and apoptotic responses. In yet another embodiment, the administration of the metal-containing texaphyrin (and in one embodiment a lanthanide-containing texaphyrin) and the zinc (II) reagent is modulated according to the increased levels in intracellular zinc in tumor cells.

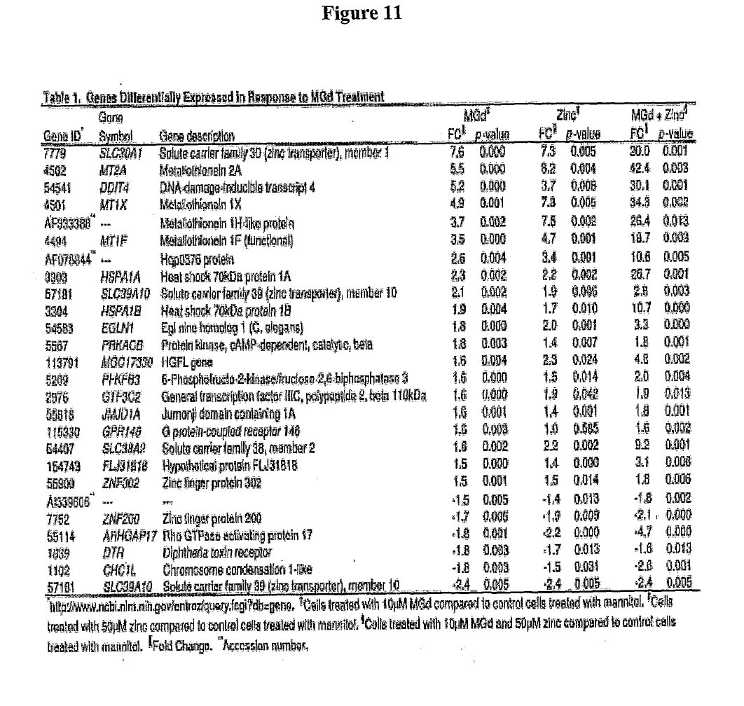

In order to better understand the molecular changes accompanying loss of zinc homeostasis prior to apoptosis, the effect of four hour treatment with MGd and/or zinc on gene expression in Ramos cultures was examined. There is an overlap in transcriptional responses to 10 μM MGd or 50 μM zinc (FIG. 11). Depending on the stringency of the criteria, up to 97% of MGd-responsive genes were also differentially expressed in the same direction in cells treated with 50 μM zinc. This indicates that MGd acts as a “zinc-mimetic” in regard to the transcriptional responses induced in Ramos cells.

Treatment with MGd or zinc or both resulted in a strong and sustained induction of MTF-1 regulated metallothionein and zinc transporter 1 (ZnT1) genes, which play roles in regulating intracellular free zinc levels, as well as HIF-1 regulated genes (e.g., PFKB3, DDIT4 and EGLN1) (FIG. 11). PFKFB3 is a kinase/phosphatase that modulates the concentration of fructose-2,6-bisphosphate, a key modulator of the glycolytic rate in proliferating cells. DDIT4 is a pro-apoptotic protein recently reported to be a negative regulator of the mammalian target of rapamycin pathway, mTOR. EGLN1 (aka., PHD2) is a prolyl hydroxylase that plays a key role in regulating HIF-1α activity by targeting HIF-1α for ubiquitin-mediated degradation. In one embodiment, the administration of an effective amount of a metal-containing texaphyrin (and in one embodiment a lanthanide-containing texaphyrin) and/or an effective amount of a zinc (II) reagent induces HIF-1 regulated genes, e.g., PFKB3, DDIT4 and EGLN1, in the target. Methods for measuring expression, induction and or activation of HIF-1 regulated genes are known in the art. In another embodiment, the induction of HIF-1 regulated genes in target cells is indicative of treatment efficacy. In yet another embodiment, the administration of the metal-containing texaphyrin (and in one embodiment a lanthanide-containing texaphyrin) and the zinc (II) reagent is modulated according to the induction of HIF-1 regulated genes in target cells. In one embodiment, the induction of HIF-1 regulated genes in target cells is indicative of treatment efficacy for compounds that act through HIF-1 regulated genes to induce cell death and/or inhibit cell proliferation of target cells. In another embodiment, the administration of such compounds is to modulate according to the induction of HIF-1 regulated genes. In yet another embodiment, an effective amount of metal-containing texaphyrin (and in one embodiment a lanthanide-containing texaphyrin) and/or an effective amount of a zinc (II) reagent is administered in combination with an effective amount of a compound that regulates HIF-1 and/or HIF-1 related genes.

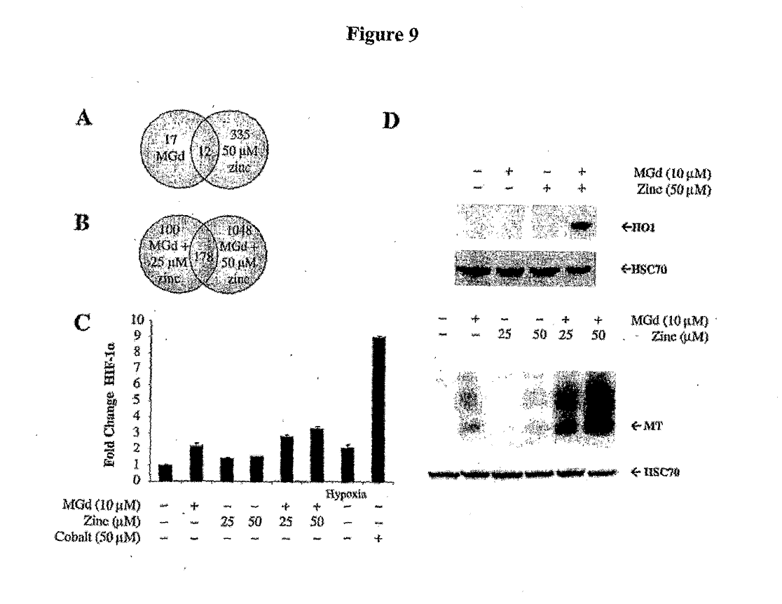

Zinc can inhibit the activity of HIF-associated hydroxylases by displacing iron from the active site of these enzymes. Greater cellular HIF-1α levels were measured by ELISA in Ramos cultures treated with either zinc or MGd (FIG. 9C). Not intending to be limited by one mechanism of action, it is proposed that MGd induces hypoxia-mimetic transcriptional responses in this system as a result of HIF-1 stabilization due to increased intracellular free zinc and/or generation of ROS. In one embodiment, the administration of an effective amount of a metal-containing texaphyrin (and in one embodiment a lanthanide-containing texaphyrin) and/or an effective amount of a zinc (II) reagent induces HIF-1α stabilization. In another embodiment, the stabilization of HIF-1α in target cells is indicative of treatment efficacy. In yet another embodiment, the administration of the metal-containing texaphyrin (and in one embodiment a lanthanide-containing texaphyrin) and/or the zinc (II) reagent is modulated according to stabilization of HIF-1α in target cells. In one embodiment, HIF-1α stabilization in target cells is indicative of treatment efficacy for compounds that act through HIF-1α stabilization to induce cell death and/or inhibit cell proliferation of target cells. In another embodiment, the administration of such compounds is modulated according to HIF-1α stabilization. Stabilization of HIF-1α in tumor cells can be measured by ELISA, Western Blot or any suitable protein assay known in the art.

The levels of transcripts under the control of MTF-1 (e.g. metallothionein family members), and under the control of HIF-1 (e.g. DDIT4) are increased synergistically in some instances by MGd and zinc treatment (Tables 1-4). These changes could contribute to the observed biological effects of the combined treatment. Indeed, the increased activation of HIF-1 would be expected to alter cellular metabolism to favor glycolysis over oxidative phosphorylation via the induction of transcripts such as PFKFB3 and PGK1. HIF-1 is often considered to be essential for tumor growth and indeed its inhibition is the subject of ongoing drug development activities. Under the appropriate conditions HIF-1 activation can have negative consequences for tumor growth by induction of targets linked to apoptosis, such as BNIP3, E2IG5, PMAIP1, and DDIT4, or through metabolic alteration of cells in the low nutrient context of the tumor microenvironment. In one embodiment, the induction of HIF-1 regulated genes is indicative of treatment efficacy. For instance, the induction of genes such as PFKFB3 and PGK1 after treatment with a metal-containing texaphyrin (and in one embodiment a lanthanide-containing texaphyrin) and the zinc (II) reagent can be an indication that the treatment will be effective. On the other hand, if genes such as BNIP3, E2IG5, PMAIP1, and DDIT4 are not induced the treatment likely will not be as effective. Methods to measure gene expression are well known in the art, including those described herein. In another embodiment, an effective amount of metal-containing texaphyrin (and in one embodiment a lanthanide-containing texaphyrin) and/or an effective amount of a zinc (II) reagent is administered in combination with an effective amount of a compound that regulates PFKFB3, PGK1, BNIP3, E2IG5, PMAIP1, or DDIT4.

In addition to the MTF-1 and HIF-1 regulated transcripts discussed above, co-treatment with MGd and zinc resulted in the expression of NRF2-regulated transcripts such as GCLM, HMOX1, and NQO3A2 which all have antioxidant response elements in their promoters. Additional transcripts such as TXNRD1, CTH, GSR, and a variety of transporters (e.g., SLC7A11) presumably involved in cellular uptake of amino acids required for glutathione synthesis are also induced. The induction of NRF-2 activity may be related to its nuclear translocation following disruption of the cytoplasmic Keap-1-NRF-2 complex. The capacity of Keap-1 to bind NRF-2 is regulated by critical cysteine residues shown to be modified under oxidative stress conditions. It has been proposed that induction of thioredoxin reductase (TXNRD1) and increased glutathione levels serves to restore Keap-2 binding of NRF2 as part of a feedback loop. Induction of NRF-2 response genes could therefore reflect the altered redox state of the cells under conditions where this enzyme is inhibited. In one embodiment, the administration of an effective amount of a metal-containing texaphyrin (and in one embodiment a lanthanide-containing texaphyrin) and/or an effective amount of a zinc (II) reagent results in the expression of NRF2-regulated transcripts such as GCLM, HMOX1, and NQO3A2 in tumor cells. In one embodiment, the expression of NRF2-regulated transcripts is indicative of treatment efficacy. Expression of NRF2-regulated transcripts can be measured by any method known in the art, including those disclosed herein. In another embodiment, an effective amount of metal-containing texaphyrin (and in one embodiment a lanthanide-containing texaphyrin) and/or an effective amount of a zinc (II) reagent is administered in combination with an effective amount of a compound that regulates NRF2 and NRF2-regulated transcripts such as GCLM, HMOX1, and NQO3A2.

In yet another embodiment, nuclear translocation of NRF2 is indicative of treatment efficacy. NRF2 nuclear translocation can be measured by any suitable method known in the art, including those described herein. Alternatively, the NRF2 and Keap-1 binding can be measured by any methods known in the art as an indicator of treatment efficacy. For instance, lower levels of NRF2-Keap-1 complex in the cytoplasm could indicate an increased in nuclear translocation of NRF2 or vice versa. In one embodiment, expression of NRF2-regulated transcripts in target cells is indicative of treatment efficacy for compounds that act through expression of NRF2-regulated transcripts to induce cell death and/or inhibit cell proliferation of target cells. In another embodiment, the administration of such compounds is modulated according to expression of NRF2-regulated transcripts, nuclear translocation of NRF2 and/or NRF2-Keap-1 complex in the cytoplasm of target cells.

Overall, the data described herein indicate that the effect of moderately increased free zinc in Ramos cells is the activation of MTF-1 and HIF-1. Induction of free zinc at higher levels increases oxidative stress, leading to the activation of NRF-2. It is one of the embodiments, to monitor levels of intracellular zinc and oxidative stress in the subjects target cells prior to and after treatment with a metal-containing texaphyrin (and in one embodiment a lanthanide-containing texaphyrin) and a zinc (II) reagent, or compounds acting through the same mechanisms, to predict treatment efficacy, modulate the administration of treatment or consider other alternatives. Another embodiment is to monitor levels of genes regulated by zinc and oxidative stress prior to and after treatment with a metal-containing texaphyrin (and in one embodiment a lanthanide-containing texaphyrin) and a zinc (II) reagent, or compounds acting through the same mechanisms, to predict treatment efficacy, modulate the administration of treatment or consider other treatment alternatives.

Another aspect involves co-administration of MGd and inhibitors of stress response pathways that are activated by MGd to increase treatment efficacy. Examples of inhibitors of stress response pathways include, but are not limited to, 17-allylamino-17-demethoxygeldanamycin (17-AAG), radicicol and actinomycin D (Dactinomycin).

### Method for Treating Cancer

Without limiting the scope of the compositions and the methods disclosed herein, the methods are used to treat several specific cancers or tumors. Cancer types include (some of which may overlap in scope), by way of example only, adrenal cortical cancer, anal cancer, aplastic anemia, bile duct cancer, bladder cancer, bone cancer, bone metastasis, adult CNS brain tumors, pediatric CNS brain metastases, brain metastases, breast cancer, Castleman Disease, cervical cancer, childhood Non-Hodgkin's lymphoma, colon and rectum cancer, endometrial cancer, esophagus cancer, Ewing's family of tumors, eye cancer, gallbladder cancer, gastrointestinal carcinoid tumors, gastrointestinal stromal tumors, gestational trophoblastic disease, hematological malignancies, Hodgkin's disease, Kaposi'sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma (adult soft tissue cancer), melanoma skin cancer, nonmelanoma skin cancer, stomach cancer, testicular cancer, thymus cancer, thyroid cancer, uterine sarcoma, vaginal cancer, vulvar cancer, and Waldenstrom's macroglobulinemia. In one embodiment, the cancers are selected from the group consisting of metastatic brain cancer, lung cancer, glioblastoma, lymphomas, leukemia, renal cell cancer (kidney cancer), head and neck cancer, breast cancer, prostrate cancer, and ovarian cancer.

Disclosed herein are methods and compositions to treat lung cancer comprising administration of an effective amount of a metal-containing texaphyrin (and in one embodiment a lanthanide-containing texaphyrin) and/or an effective amount of a zinc (II) reagent. Treatment options for lung cancer include (which can be provided to a patient in conjunction with administration of an effective amount of a metal-containing texaphyrin (and in one embodiment a lanthanide-containing texaphyrin) and/or an effective amount of a zinc (II) reagent), by way of example only, surgery, immunotherapy, radiation therapy, chemotherapy, photodynamic therapy, or a combination thereof. Some possible surgical options for treatment of lung cancer are a segmental or wedge resection, a lobectomy, or a pneumonectomy. Radiation therapy may be external beam radiation therapy or brachytherapy.

Disclosed herein are methods and compositions to treat CNS neoplasms comprising administration of an effective amount of a metal-containing texaphyrin (and in one embodiment a lanthanide-containing texaphyrin) and/or an effective amount of a zinc (II) reagent. Treatment options for CNS neoplasms include (which can be provided to a patient in conjunction with administration of an effective amount of a metal-containing texaphyrin (and in one embodiment a lanthanide-containing texaphyrin) and/or an effective amount of a zinc (II) reagent), by way of example only, surgery, radiation therapy, immunotherapy, hyperthermia, gene therapy, chemotherapy, and combination of radiation and chemotherapy. Doctors also may prescribe steroids to reduce the swelling inside the CNS.

Disclosed herein are methods to treat kidney cancer comprising administration of an effective amount of a metal-containing texaphyrin (and in one embodiment a lanthanide-containing texaphyrin) and/or an effective amount of a zinc (II) reagent. Kidney cancer (also called renal cell cancer or renal adenocarcinoma) is a disease in which malignant cells are found in the lining of tubules in the kidney. Treatment options for kidney cancer include (which can be provided to a patient in conjunction with administration of an effective amount of a metal-containing texaphyrin (and in one embodiment a lanthanide-containing texaphyrin) and/or an effective amount of a zinc (II) reagent), by way of example only, surgery, radiation therapy, chemotherapy and immunotherapy. Some possible surgical options to treat kidney cancer include, by way of example only, partial nephrectomy, simple nephrectomy and radical nephrectomy. Radiation therapy may be external beam radiation therapy or brachytherapy. Stem cell transplant may be used to treat kidney cancer.

In one embodiment disclosed herein are methods to treat lymphoma comprising administration of an effective amount of a metal-containing texaphyrin (and in one embodiment a lanthanide-containing texaphyrin) and/or an effective amount of a zinc (II) reagent. Treatment options for lymphoma include (which can be provided to a patient in conjunction with administration of an effective amount of a metal-containing texaphyrin (and in one embodiment a lanthanide-containing texaphyrin) and/or an effective amount of a zinc (II) reagent), by way of example only, chemotherapy, immunotherapy, radiation therapy and high-dose chemotherapy with stem cell transplant. Radiation therapy may be external beam radiation therapy or brachytherapy.

Disclosed herein are methods for treating breast cancer comprising administration of an effective amount of a metal-containing texaphyrin (and in one embodiment a lanthanide-containing texaphyrin) and/or an effective amount of a zinc (II) reagent. Treatment options for breast cancer include (which can be provided to a patient in conjunction with administration of an effective amount of a metal-containing texaphyrin (and in one embodiment a lanthanide-containing texaphyrin) and/or an effective amount of a zinc (II) reagent), by way of example only, surgery, immunotherapy, radiation therapy, chemotherapy, endocrine therapy, or a combination thereof. A lumpectomy and a mastectomy are two possible surgical procedures available for breast cancer patients.

Disclosed herein are methods for treating ovarian cancer, comprising administration of an effective amount of a metal-containing texaphyrin (and in one embodiment a lanthanide-containing texaphyrin) and/or an effective amount of a zinc (II) reagent. Treatment options for ovarian cancer include (which can be provided to a patient in conjunction with administration of an effective amount of a metal-containing texaphyrin (and in one embodiment a lanthanide-containing texaphyrin) and/or an effective amount of a zinc (II) reagent), by way of example only, surgery, immunotherapy, chemotherapy, hormone therapy, radiation therapy, or combinations thereof. Some possible surgical procedures include debulking, and a unilateral or bilateral oophorectomy and/or a unilateral or bilateral salpigectomy.

Disclosed herein are methods for treating cervical cancer, comprising administration of an effective amount of a metal-containing texaphyrin (and in one embodiment a lanthanide-containing texaphyrin) and/or an effective amount of a zinc (II) reagent. Treatment options for cervical cancer include (which can be provided to a patient in conjunction with administration of an effective amount of a metal-containing texaphyrin (and in one embodiment a lanthanide-containing texaphyrin) and/or an effective amount of a zinc (II) reagent), by way of example only, surgery, immunotherapy, radiation therapy and chemotherapy. Some possible surgical options are cryosurgery, a hysterectomy, and a radical hysterectomy. Radiation therapy for cervical cancer patients includes external beam radiation therapy or brachytherapy.

Disclosed herein are methods to treat prostate cancer, comprising administration of an effective amount of a metal-containing texaphyrin (and in one embodiment a lanthanide-containing texaphyrin) and/or an effective amount of a zinc (II) reagent. Treatment options for prostate cancer include (which can be provided to a patient in conjunction with administration an effective amount of a metal-containing texaphyrin (and in one embodiment a lanthanide-containing texaphyrin) and/or an effective amount of a zinc (II) reagent), by way of example only, surgery, immunotherapy, radiation therapy, cryosurgery, hormone therapy, and chemotherapy. Possible surgical procedures to treat prostate cancer include, by way of example only, radical retropubic prostatectomy, a radical perineal prostatectomy, and a laparoscopic radical prostatectomy. Some radiation therapy options are external beam radiation, including three dimensional conformal radiation therapy, intensity modulated radiation therapy, and conformal proton beam radiation therapy. Brachytherapy (seed implantation or interstitial radiation therapy) is also an available method of treatment for prostate cancer. Cryosurgery is another possible method used to treat localized prostate cancer cells. Hormone therapy, also called androgen deprivation therapy or androgen suppression therapy, may be used to treat prostate cancer. Several methods of this therapy are available including an orchiectomy in which the testicles, where 90% of androgens are produced, are removed. Another method is the administration of luteinizing hormone-releasing hormone (LHRH) analogs to lower androgen levels. The LHRH analogs available include leuprolide, nafarelin, goserelin, triptorelin, and histrelin. An LHRH antagonist may also be administered, such as abarelix. Treatment with an antiandrogen agent, which blocks androgen activity in the body, is another available therapy. Such agents include flutamide, bicalutamide, and nilutamide. This therapy is typically combined with LHRH analog administration or an orchiectomy, which is termed a combined androgen blockade (CAB). Chemotherapy may be appropriate where a prostate tumor has spread outside the prostate gland and hormone treatment is not effective. Anti-cancer drugs may be administered to slow the growth of prostate cancer, reduce symptoms and improve the quality of life.

Disclosed herein are methods for treating leukemia, comprising administration of an effective amount of a metal-containing texaphyrin (and in one embodiment a lanthanide-containing texaphyrin) and/or an effective amount of a zinc (II) reagent. Treatment options for leukemia include (which can be provided to a patient in conjunction with administration of an effective amount of a metal-containing texaphyrin (and in one embodiment a lanthanide-containing texaphyrin) and/or an effective amount of a zinc (II) reagent), by way of example only, immunotherapy, radiation therapy, chemotherapy, bone marrow or peripheral blood stem cell transplantation, or a combination thereof. Radiation therapy includes external beam radiation and may have side effects. Anti-cancer drugs may be used in chemotherapy to treat leukemia. Monoclonal antibody therapy may be used to treat AML patients. Small molecules or radioactive chemicals may be attached to these antibodies before administration to a patient in order to provide a means of killing leukemia cells in the body. The monoclonal antibody, gemtuzumab ozogamicin, which binds CD33 on AML cells, may be used to treat AML patients unable to tolerate prior chemotherapy regimens. Bone marrow or peripheral blood stem cell transplantation may be used to treat AML patients. Some possible transplantation procedures are an allogenic or an autologous transplant.

Disclosed herein are methods and compositions to treat head and neck cancer, comprising administration of an effective amount of a metal-containing texaphyrin (and in one embodiment a lanthanide-containing texaphyrin) and/or an effective amount of a zinc (II) reagent. Treatment options for head and neck cancer include (which can be provided to a patient in conjunction with administration of the high-purity compositions of Formula I), by way of example only, surgery, radiation, chemotherapy, combined modality therapy, gene therapy, either alone or in combination thereof.

### Pharmaceutical Compositions

Metal-containing texaphyrin (and in one embodiment a lanthanide-containing texaphyrin)s are typically administered in the form of pharmaceutical compositions. Zinc (II) reagents are also administered in the form of pharmaceutical compositions. When metal-containing texaphyrin (and in one embodiment a lanthanide-containing texaphyrin)s and zinc (II) reagents are used in combination, both components may be mixed into a preparation or both components may be Formulated into separate preparations to use them in combination at the same time. This application therefore provides pharmaceutical compositions that contain, as the active ingredient, metal-containing texaphyrin (and in one embodiment a lanthanide-containing texaphyrin)s or a pharmaceutically acceptable salt AND/or coordination complex thereof, and one or more pharmaceutically acceptable excipients, carriers, including inert solid diluents and fillers, diluents, including sterile aqueous solution and various organic solvents, permeation enhancers, solubilizers and adjuvants. This application also provides pharmaceutical compositions that contain, as the active ingredient, zinc (II) reagents or a pharmaceutically acceptable salt AND/or coordination complex thereof, and one or more pharmaceutically acceptable excipients, carriers, including inert solid diluents and fillers, diluents, including sterile aqueous solution and various organic solvents, permeation enhancers, solubilizers and adjuvants. This application further provides pharmaceutical compositions that contain, as the active ingredient, metal-containing texaphyrin (and in one embodiment a lanthanide-containing texaphyrin)s or a pharmaceutically acceptable salt AND/or coordination complex thereof, zinc (II) reagents or a pharmaceutically acceptable salt AND/or coordination complex thereof, and one or more pharmaceutically acceptable excipients, carriers, including inert solid diluents and fillers, diluents, including sterile aqueous solution and various organic solvents, permeation enhancers, solubilizers and adjuvants. In one embodiment, the metal-containing texaphyrin is motexafin gandolinium and the zinc (II) reagent is zinc acetate. In another embodiment, an effective amount of metal-containing texaphyrin (and in one embodiment a lanthanide-containing texaphyrin)s and an effective amount of a zinc (II) reagent may be administered in combination with other therapeutic agents. Such compositions are prepared in a manner well known in the pharmaceutical art.

One embodiment described herein is a packaged product of Formula (I) for intravenous drug use to a human subject wherein the packaging will not significantly absorb, react with, or otherwise adversely affect the drug or other excipients or components used in intravenous delivery during storage of the system prior to its use. In a further embodiment described herein are packaged products of Formula (I) for intravenous delivery, comprising a texaphyrin metal complex of Formula (I). The foregoing and other objectives are achieved by providing light protective materials and a substantially deoxygenated environment to prevent degradation to Formula (I) prior to use. Such light protective materials include an outer packaging that is opaque and an inner package that comprises a transparent, non-tinted material, such as glass. The packaging of Formula (I) for intravenous use is dependent on the form of the drug, see FIG. 1. In one embodiment, Formula (I) may be packaged in liquid form. In another embodiment, Formula (I) may be packaged in powder form with reconstituting solution.

Suitable storage-stabilized Formulations of Formula (I) include a solution of Formula (I) in water and acetic acid. In one embodiment, the storage-stabilized Formulation should have a pH of 5.4. In other embodiments, the storage-stabilized Formulation should have a pH between about 4.5-5.5, about 5.0-5.9 or about 4.9-5.9. In another embodiment the concentration of Formula (I) in the storage-stabilized Formulation is between 2.5 mg/mL and about 3.0 mg/mL; in a further embodiment the concentration of Formula (I) is about 2.5 mg/mL.

In further or alternative embodiments, storage-stabilized Formulation contains an isotonic agent, which can include electrolytes and/or non-electrolytes. Non-limiting examples of electrolytes includes sodium chloride, potassium chloride, dibasic sodium phosphate, sodium gluconate and combinations thereof. Non-limiting examples of non-electrolytes includes saccharides and polyhydric alcohols; further examples include mannitol, sorbitol, glucose, dextrose, glycerol, xylitol, fructose, maltose, mannose, glycerin, propylene glycol, and combinations thereof. In still further embodiments, the storage-stabilized Formulation comprises a buffer, an anti-crystallizing agent, and/or a preservative. Buffering agents aid in stabilizing pH. Anti-crystallizing agents aid in stabilizing the concentration of the solution. Preservatives aid in preventing the growth of micro-organisms, and include by way of example only, methyl paraben, propyl paraben, benzyl alcohol, sodium hypochlorite, phenoxy ethanol and/or propylene glycol. In one, the storage-stabilized Formulation does not contain an oxidizing agent other than Formula (I) and oxygen. Oxidizing agents promote degradation of the compound of Formula (I).

Compounds of Formula (I) can be synthesized by procedures outlined in U.S. Pat. Nos. 4,935,498, 5,252,720, 5,801,229, 5,451,576, 5,569,759, and 6,638,924, and U.S. patent application Ser. No. 11/235,475 filed on Sep. 26, 2005, the disclosures of which are incorporated by reference in their entirety. Compounds of Formula (I) can be Formulated into an intravenously-acceptable pharmaceutical Formulation, and stored as such a Formulation, as described in U.S. Pat. Nos. 6,919,327 and 6,638,924, and U.S. patent application Ser. No. 11/241,549 filed on Sep. 30, 2005, the disclosures of which are incorporated by reference in their entirety.

### Administration

An effective amount of a metal-containing texaphyrin (and in one embodiment a lanthanide-containing texaphyrin) and/or an effective amount of a zinc (II) reagent may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer. Presently, the metal-containing texaphyrin (and in one embodiment a lanthanide-containing texaphyrin) and the zinc (II) reagent are administered in combination. This administration in combination can include simultaneous administration of the two agents in the same dosage form, simultaneous administration in separate dosage forms, and separate administration. That is, metal-containing texaphyrin (and in one embodiment a lanthanide-containing texaphyrin) the zinc (II) reagent can be Formulated together in the same dosage form and administered simultaneously. Alternatively, metal-containing texaphyrin (and in one embodiment a lanthanide-containing texaphyrin) and the zinc (II) reagent can be simultaneously administered, wherein both the agents are present in separate Formulations. In another alternative, the zinc (II) reagent can be administered just followed by the metal-containing texaphyrin (and in one embodiment a lanthanide-containing texaphyrin), or vice versa. In the separate administration protocol, the metal-containing texaphyrin (and in one embodiment a lanthanide-containing texaphyrin) and the zinc (II) reagent may be administered a few minutes apart, or a few hours apart, or a few days apart.

One mode for administration is parental, particularly by injection. The forms in which the novel compositions of the disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

It has been discovered that texaphyrins have a tendency to aggregate in aqueous solution, which potentially decreases their solubility. Aggregation (self-association) of polypyrrolic macrocyclic compounds, including porphyrins, sapphyrins, texaphyrins, and the like, is a common phenomenon in water solution as the result of strong intermolecular van der Waals attractions between these flat aromatic systems. Aggregation may significantly alter the photochemical characteristics of the macrocycles in solution, which is shown by large spectral changes, decrease in extinction coefficient, etc.

It has been found that addition of a carbohydrate, saccharide, polysaccharide, or polyuronide to the Formulation decreases the tendency of the texaphyrin to aggregate, thus increasing the solubility of the texaphyrin in aqueous media. Anti-aggregation agents are sugars, in particular mannitol, dextrose or glucose, mannitol of about 2-8% concentration, and about 5% concentration. These aqueous solutions are especially suitable for intravenous, intramuscular, subcutaneous and intraperitoneal administration. In this connection, the sterile aqueous media that can be employed will be known to those of skill in the art in light of the present disclosure.

Prolonged absorption of the injectable compositions can be brought about by the use in the compositions of agents delaying absorption, for example, aluminum monostearate and gelatin. These particular aqueous solutions are especially suitable for intra-arterial, intravenous, intramuscular, subcutaneous and intraperitoneal administration. In this connection, sterile aqueous media that can be employed will be known to those skilled in the art in light of the present disclosure.

Sterile injectable solutions are prepared by incorporating the metal-containing texaphyrin (and in one embodiment a lanthanide-containing texaphyrin) AND/or the zinc (II) reagent in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the methods of preparation are vacuum-drying and freeze-drying techniques which yield a powder of the active ingredient plus any additional desired ingredient from a previously sterile-filtered solution thereof.

The metal-containing texaphyrin (and in one embodiment a lanthanide-containing texaphyrin) AND/or the zinc (II) reagent may be impregnated into a stent by diffusion, for example, or coated onto the stent such as in a gel form, for example, using procedures known to one of skill in the art in light of the present disclosure.

Oral administration is another route for administration of the metal-containing texaphyrin (and in one embodiment a lanthanide-containing texaphyrin) and the zinc (II) reagent of this application. Oral administration via capsule or enteric coated tablets, or the like, prevent degradation of the metal-containing texaphyrin (and in one embodiment a lanthanide-containing texaphyrin) AND/or the zinc (II) reagent of the disclosure in the stomach. In making the pharmaceutical compositions that include at least one metal-containing texaphyrin (and in one embodiment a lanthanide-containing texaphyrin) AND/or at least one zinc (II) reagent, the active ingredient is usually diluted by an excipient and/or enclosed within such a carrier that can be in the form of a capsule, sachet, paper or other container. When the excipient serves as a diluent, in can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The Formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions can be Formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix Formulations. Examples of controlled release systems are given in U.S. Pat. Nos. 3,845,770; 4,326,525; 4,902,514; and 5,616,345. Another Formulation for use in the methods disclosed employs transdermal delivery devices (“patches”). Such transdermal patches may be used to provide continuous or discontinuous infusion of the Motexafin Gadolinium in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions can be Formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The metal-containing texaphyrin (and in one embodiment a lanthanide-containing texaphyrin) is effective over a wide dosage range and is generally administered in a pharmaceutically effective amount. For oral administration, each dosage unit contains from 10 mg to 2 g of the metal-containing texaphyrin (and in one embodiment a lanthanide-containing texaphyrin), and for parenteral administration, from 10 to 700 mg of the metal-containing texaphyrin (and in one embodiment a lanthanide-containing texaphyrin), or about 350 mg. The zinc (II) reagent is effective over a wide dosage range and is generally administered in a pharmaceutically effective amount. For oral administration, each dosage unit contains from 40-100 μmol/kg of the zinc (II) reagent, It will be understood, however, that the amount of the metal-containing texaphyrin (and in one embodiment a lanthanide-containing texaphyrin) and/or zinc (II) reagent actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preFormulation composition containing a homogeneous mixture of the metal-containing texaphyrin (and in one embodiment a lanthanide-containing texaphyrin) AND/or the zinc (II) reagent. When referring to these preFormulation compositions as homogeneous, it is meant that the active ingredient is dispersed evenly throughout the composition so that the composition may be readily subdivided into equally effective unit dosage forms such as tablets, pills and capsules.

The tablets or pills presented herein may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. The compositions are administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, orally or nasally, from devices that deliver the Formulation in an appropriate manner.

### Activation Means

The metal-containing texaphyrin (and in one embodiment a lanthanide-containing texaphyrin) and the zinc (II) reagent will be administered in a therapeutically effective amount, employing a method of administration and a pharmaceutical Formulation as discussed above, and optionally a means of activation of the metal-containing texaphyrin (and in one embodiment a lanthanide-containing texaphyrin) (through a therapeutic energy or agent) as is known in the art. The therapeutic energy or agent to be used includes photodynamic therapy, radiation sensitization, chemotherapy, sonodynamic therapy, and neutron bombardment. The specific dose will vary depending on the dosing regimen to be followed, and the particular therapeutic energy or agent with which it is administered. Such dose can be determined by methods known in the art or as described herein.

Dosages: The specific dose will vary depending on the dosing regimen to be followed, and the particular therapeutic energy or agent with which it is administered, employing dosages within the range of about 0.01 mg/kg/treatment up to about 100 mg/kg/treatment, or about 0.1 mg/kg/treatment to about 50 mg/kg/treatment. It will be appreciated by one skilled in the art, however, that there are specific differences in the most effective dosimetry depending on the ligands chosen, because of the wide range of properties available, such as solubilities, lipophilicity properties, lower toxicity, and improved stability.

### Administration for Photodynamic Therapy

The metal-containing texaphyrin (and in one embodiment a lanthanide-containing texaphyrin) and the zinc (II) reagent may be administered in solution, optionally in 5% mannitol USP. Dosages of about 1.0-2.0 mg/kg to about 4.0-7.0 mg/kg, or 3.0 mg/kg, are employed, although in some cases a maximum tolerated dose may be higher, for example about 5 mg/kg. The metal-containing texaphyrin (and in one embodiment a lanthanide-containing texaphyrin) and the zinc (II) reagent are administered by intravenous injection, followed by a waiting period of from as short a time as several minutes or about 3 hours to as long as about 72 or 96 hours (depending on the treatment being effected) to facilitate intracellular uptake and clearance from the plasma and extracellular matrix prior to the administration of photoirradiation.

Dose levels for certain uses may range from about 0.05 mg/kg to about 20 mg/kg administered in single or multiple doses (e.g. before each fraction of radiation). The lower dosage range would be applicable for intra-arterial injection or for impregnated stents.

The co-administration of a sedative (e.g., benzodiazapenes) and narcotics/analgesics are sometimes recommended prior to light treatment along with topical administration of a local anesthetic, for example Emla cream (lidocaine, 2.5% and prilocaine, 2.5%) under an occlusive dressing. Other intradermal, subcutaneous and topical anesthetics may also be employed as necessary to reduce discomfort. Subsequent treatments can be provided after approximately 21 days.

The optimum length of time following administration of Motexafin Gadolinium until light treatment can vary depending on the mode of administration, the form of administration, and the type of target tissue. Typically, Motexafin Gadolinium persists for a period of minutes to hours, depending on the Formulation, the dose, the infusion rate, as well as the type of tissue and tissue size.

### Administration for Chemosensitization

The metal-containing texaphyrin (and in one embodiment a lanthanide-containing texaphyrin) and the zinc (II) reagent may be administered before, at the same time, or after administration of one or more chemotherapeutic drugs. Motexafin Gadolinium may be administered as a single dose, or it may be administered as two or more doses separated by an interval of time. Motexafin Gadolinium may be administered concurrently with, or from about one minute to about 12 hours following, administration of a chemotherapeutic drug, from about 5 min to about 5 hr, or about 4 to 5 hr. The dosing protocol may be repeated, from one to three times, for example. A time frame that has been successful in vivo is administration of Motexafin Gadolinium about 5 min and about 5 hr after administration of a chemotherapeutic agent, with the protocol being performed once per week for three weeks. Administration may be intra-arterial injection, intravenous, intraperitoneal, intramuscular, subcutaneous, intrathecally, oral, topical, or via a device such as a stent.

Administering Motexafin Gadolinium and a chemotherapeutic drug to the subject may be prior to, concurrent with, or following vascular intervention. The method may begin at a time roughly accompanying a vascular intervention, such as an angioplastic procedure, for example. Multiple or single treatments prior to, at the time of, or subsequent to the procedure may be used. “Roughly accompanying a vascular intervention” refers to a time period within the ambit of the effects of the vascular intervention. Typically, an initial dose of Motexafin Gadolinium and chemotherapeutic drug will be within 6-12 hours of the vascular intervention, within 6 hours thereafter. Follow-up dosages may be made at weekly, biweekly, or monthly intervals. Design of particular protocols depends on the individual subject, the condition of the subject, the design of dosage levels, and the judgment of the attending practitioner.

### Administration for Radiation Sensitization

Motexafin Gadolinium where the metal is gadolinium is typically administered in a solution containing 2 mM optionally in 5% mannitol USP/water (sterile and non-pyrogenic solution). Dosages of 0.1 mg/kg up to as high as about 29.0 mg/kg have been delivered, or about 3.0 to about 15.0 mg/kg (for volume of about 90 to 450 mL) may be employed, optionally with pre-medication using anti-emetics when dosing above about 6.0 mg/kg. Motexafin Gadolinium is administered via intravenous injection over about a 5 to 10 minute period, followed by a waiting period of about 2 to 5 hours to facilitate intracellular uptake and clearance from the plasma and extracellular matrix prior to the administration of radiation.

When employing whole brain radiation therapy, a course of 30 Gy in ten (10) fractions of radiation may be administered over consecutive days excluding weekends and holidays. In the treatment of brain metastases, whole brain megavolt radiation therapy is delivered with ⁶⁰Co teletherapy or a .gtoreq.4 MV linear accelerator with isocenter distances of at least 80 cm, using isocentric techniques, opposed lateral fields and exclusion of the eyes. A minimum dose rate at the midplane in the brain on the central axis is about 0.5 Gy/minute.

Motexafin Gadolinium used as radiation sensitizers may be administered before, or at the same time as, or after administration of the ionizing radiation. Motexafin Gadolinium may be administered as a single dose, as an infusion, or it may be administered as two or more doses separated by an interval of time. Where Motexafin Gadolinium is administered as two or more doses, the time interval between Motexafin Gadolinium administrations may be from about one minute to a number of days, or from about 5 min to about 1 day, or from about 4 to 5 hr. The dosing protocol may be repeated, from one to ten or more times, for example. Dose levels for radiation sensitization may range from about 0.05 mg/kg to about 20 mg/kg administered in single or multiple doses (e.g. before each fraction of radiation). The lower dosage range is typical for intra-arterial injection or for impregnated stents.

Administration may be intra-arterial injection, intravenous, intraperitoneal, intramuscular, subcutaneous, oral, topical, or via an impregnated or coated device such as a stent, for example, or an artery-inserted cylindrical polymer. In one aspect, a patient having restenosis or at risk for restenosis is administered a dose of Motexafin Gadolinium at intervals with each dose of radiation.

Administering Motexafin Gadolinium to the subject may be prior to, concurrent with, or following vascular intervention, and the intervention is followed by radiation. The method may begin prior to, such as about 24-48 hours prior to, or at a time roughly accompanying vascular intervention, for example. Multiple or single treatments prior to, at the time of, or subsequent to the procedure may be used. “Roughly accompanying the vascular intervention” refers to a time period within the ambit of the effects of the vascular intervention. Typically, an initial dose Motexafin Gadolinium and radiation will be within 1-24 hours of the vascular intervention, or within about 5-24 hours thereafter. Follow-up dosages may be made at weekly, biweekly, or monthly intervals. Design of particular protocols depends on the individual subject, the condition of the subject, the design of dosage levels, and the judgment of the attending practitioner.

### Administration for Sonodynamic Therapy:

The use of texaphyrins in sonodynamic therapy is described in U.S. patent application Ser. No. 09/111,148, which is incorporated herein by reference. Texaphyrin is administered before administration of the ultrasound. The texaphyrin may be administered as a single dose, or it may be administered as two or more doses separated by an interval of time. Parenteral administration is typical, including by intravenous and interarterial injection. Other common routes of administration can also be employed.

Ultrasound is generated by a focused array transducer driven by a power amplifier. The transducer can vary in diameter and spherical curvature to allow for variation of the focus of the ultrasonic output. Commercially available therapeutic ultrasound devices may be employed in the practice of what is claimed in the application. The duration and wave frequency, including the type of wave employed may vary, the typical duration of treatment will vary from case to case within the judgment of the treating physician. Both progressive wave mode patterns and standing wave patterns have been successful in producing cavitation of diseased tissue. When using progressive waves, the second harmonic can advantageously be superimposed onto the fundamental wave.

Various types of ultrasound employed presently are ultrasound of low intensity, non-thermal ultrasound, i.e., ultrasound generated within the wavelengths of about 0.1 MHz and 5.0 MHz and at intensities between about 3.0 and 5.0 W/cm².

### Administration for Neutron Capture Therapy

The use of metallotexaphyrins in neutron capture therapy is described in U.S. patent application Ser. No. 60/229,366, entitled “Agents for Neutron Capture Therapy”, filed on Aug. 30, 2000, which is incorporated herein in its entirety by reference. The metallotexaphyrin is administered before administration of the neutron beam. It may be administered as a single dose, or it may be administered as two or more doses separated by an interval of time. Parenteral administration is typical, including by intravenous and interarterial injection. Other common routes of administration can also be employed.

The following examples are included to demonstrate embodiments. It should be appreciated by those of skill in the art that the techniques disclosed in the examples which follow represent techniques discovered by the Applicant to function well in the practice of what is claimed in this application, and thus can be considered to constitute modes for its practice. However, those of skill in the art should, in light of the present disclosure, appreciate that many changes can be made in the specific embodiments which are disclosed and still obtain a like or similar result without departing from the spirit and scope of the disclosure.

### EXAMPLES

### Example 1

### Cells and Cell Culture

Ramos, Raji, DB B-cell lymphoma, DHL-4 and HF-1 cell lines were cultured in a 5% CO₂ incubator at 37° C. at a density between 0.2 and 1×10⁶ cells/mL. Motexafin gadolinium (MGd) was prepared as a 2 mM (2.3 mg/mL) Formulation in 5% aqueous mannitol. Zinc acetate (Zn(OAc)₂) and cobalt acetate (Aldrich Chemical, Milwaukee, Wis.) were used as 2 mM Formulations in 5% aqueous mannitol.

### Example 2

### Gene Expression

Methods: A549 human lung cancer cells (6.5×10⁵ cells per T-162 flask in 45 mL complete RPMI 1640 medium) were seeded 10 days prior to treatment of non-cycling plateau phase cultures with MGd. At 4, 12, or 24 hours prior to RNA isolation, MGd (50 μM final concentration) or control (5% mannitol) solution was added to the cultures. Each time course experiment was performed in triplicate. After incubation, all cultures were washed once with PBS and total RNA was subjected to analysis on Affymetrix U133A microarrays, designed to interrogate the relative abundance of over 15,000 human genes.

We used Microarray Suite version 5.0 software (Affymetrix) to generate raw gene expression scores and normalized the relative hybridization signal from each experiment. All gene expression scores were set to a minimum value of one hundred in order to minimize noise associated with less robust measurements of rare transcripts. The permutation-based significance analysis of microarrays (SAM) was used to determine genes differentially expressed in response to MGd treatment compared to the control 5% mannitol-treated cultures (0.125% final) at each time point. We report all genes at least 2-fold differentially expressed with a less than a 1% False Discovery Rate (FDR) in response to MGd treatment (FIG. 10). The use of lower fold change cut-offs (down to 1.01-fold) in SAM analysis did not identify any additional differentially expressed genes.

Results: To assess the effects of drug on gene expression profiles, total cellular RNA was isolated from plateau phase A549 cultures treated with 50 μM MGd for 4, 12, and 24 hours in triplicate and analyzed on oligonucleotide microarrays. Eleven genes showed at least a two-fold differential expression in response to MGd treatment (averaged across all time frames) that reached statistical significance by SAM analysis (FIG. 10). The most prominent consequence of MGd treatment was the up-regulation of various metallothionein isoform transcripts at all time points. In fact, 10 of the 11 transcripts listed in FIG. 10 are metallothionein-related. The remaining transcript, hbc647, was also up-regulated by MGd treatment at all three time points. This cDNA is located 5-kb downstream of the zinc transporter 1 (ZnT1) gene and is likely to comprise part of the 3′-UTR of this gene. Northern blot analysis confirmed the induction of metallothionein gene family members and ZnT1 (Top, FIG. 1B). These transcripts are under the control of metal-response element-binding transcription factor-1 (MTF-1) (FIG. 1C).

### Example 3

### Metallothionein Induction by MGd Compared to Free Gadolinium Acetate

Methods: Northern Blot Analysis—Plateau phase cultures of A549 cells were prepared as described above, except that T-25 flasks were used, and the number of cells initially plated scaled accordingly. Cultures were treated with 50 μM MGd, 5 μM Gd(OAc)₃ or 5% mannitol for 4 hours, whereupon cultures were washed twice with PBS and RNA harvested as above. Alternatively, 7-day plateau phase A549 or PC3 cultures were treated with 50 μM MGd, 50-100 μM Zn(OAc)₂, 50 μM CdCl₂ or 5% mannitol for 24 hours (A549) or 4 hours (PC3) prior to washing and RNA analysis. Exponential phase Ramos cultures were treated for 6 hours. Northern blots were conducted and analyzed. Radio labeled metallothionein probe, designed to bind to a 113-bp region of 3′-UTR sequences from multiple metallothionein gene family members, was generated using 5′-ATGGACCCCAACTGCTCCTG-3′ (forward) and 5′-GGGCAGCAGGAGCAGCAGCT-3′ (reverse) PCR primers and the NEBlot kit (New England Biolabs, Inc.). Radio labeled ZnT1 probe was generated in a similar fashion using 5′-TGCTGGAAGCAGAATCATTG-3′ (forward) and 5′-TGCTAACTGCTGGGGTCTTT-3′ (reverse) primers.

Results: Studies were performed to determine whether the metallothionein transcript up-regulation observed in the microarray analysis could be a consequence of free gadolinium(III) cation, released from MGd into the cell culture medium. HPLC analysis of MGd stability in medium obtained from plateau phase cultures indicated that the drug appeared to be stable, with an apparent loss of only 4% after 24 hours. Since the microarray analysis indicated strong transcript up-regulation after 4 hours of treatment with drug, this time interval was selected for further investigation of RNA transcript levels. RNA was harvested from plateau phase cultures treated with control vehicle, 50 μM MGd, or 5 μM Gd(OAc)3 for 4 hours. Northern blot analysis indicated metallothionein gene family members and ZnT1 were induced only in cultures treated with MGd (bottom, FIG. 1B). Thus, even assuming a 10% loss of drug over the initial 4 hour incubation period, the resulting release of Gd(III) ion into the culture medium would be insufficient to induce the observed metallothionein and ZnT1 transcript levels.

### Example 4

### MGd Increases Sensitivity of Cells to Zinc Acetate

Methods: Cellular Viability. Cell viability was determined by using propidium iodide (PI) flow cytometric analysis. Cells from plateau phase cultures grown in T-25 flasks were harvested as described above, except that cells present in the growth medium and wash solution were isolated by centrifugation and included in the analysis. Cells were resuspended in 1 mL PBS, an aliquot of 3×10⁵ cells transferred to a 4 mL tube, and the cells isolated by centrifugation. Cell pellets were resuspended in PBS supplemented with 2 μg/mL propidium iodide (Sigma), incubated for 5 minutes at ambient temperature, and subjected to flow cytometric analysis. Flow cytometry was performed on a FACSCalibur instrument and data were analyzed using the CellQuest Pro software package (BD Biosciences).

Results: The findings in Example 3 indicate that MGd treatment might alter cellular response to metal cations. This could be relevant since the levels of zinc available to cultured cancer cells and to cancer cells in vivo may not directly coincide in all instances. To examine this possibility, A549 cultures were treated with Zn(OAc)₂ (100 μM) or CdCl₂ (50 μM) for 24 hours in the presence or absence of 50 μM MGd. Treatment with Zn(OAc)₂ alone had no effect on cellular viability, as assessed by propidium iodide exclusion (FIG. 2A). By contrast, viability dropped from ca. 90% to ca. 55% after treatment with Zn(OAc)₂ and MGd. This is dependent on treatment time since there are limited effects on cellular viability at 4 hours and only intermediate effects at 12 hours relative to 24 hour treatments (Supplementary FIG. 1). Conversely, viability after CdCl₂ treatment, ca. 53%, was increased to ca. 76% by co-incubation with MGd.

### Example 5

### Metallothionein Induction by Zinc and Cadmium Treatment in the Presence and Absence of MGd

Methods: Northern Blot Analysis—Plateau phase cultures of A549 cells were prepared as described above, except that T-25 flasks were used, and the number of cells initially plated scaled accordingly. Cultures were treated with 50 μM MGd, 5 μM Gd(OAc)₃ or 5% mannitol for 4 hours, whereupon cultures were washed twice with PBS and RNA harvested as above. Alternatively, 7-day plateau phase A549 or PC3 cultures were treated with 50 μM MGd, 50-100 μM Zn(OAc)₂, 50 μM CdCl₂ or 5% mannitol for 24 hours (A549) or 4 hours (PC3) prior to washing and RNA analysis. Exponential phase Ramos cultures were treated for 6 hours. Northern blots were conducted and analyzed. Radio labeled metallothionein probe, designed to bind to a 113-bp region of 3′-UTR sequences from multiple metallothionein gene family members, was generated using 5′-ATGGACCCCAACTGCTCCTG-3′ (forward) and 5′-GGGCAGCAGGAGCAGCAGCT-3′ (reverse) PCR primers and the NEBlot kit (New England Biolabs, Inc.). Radio labeled ZnT1 probe was generated in a similar fashion using 5′-TGCTGGAAGCAGAATCATTG-3′ (forward) and 5′-TGCTAACTGCTGGGGTCTTT-3′ (reverse) primers.

Results: RNA harvested from surviving A549 cells, treated as above, was analyzed for metallothionein transcript induction. Treatment with Zn(OAc)₂, CdCl₂, or MGd resulted in significant increase in the levels of these RNA transcripts (FIG. 2B). Interestingly, co-treatment with Zn(OAc)₂ and MGd led to a synergistic increase in the levels of metallothionein transcripts (FIG. 2B). Treatment with cadmium led to high levels of metallothionein transcription, in the presence or absence of MGd.

### Example 6

### Intracellular Free Zinc is Elevated in MGd-Treated Cells

Methods: The concentration of intracellular free zinc was assessed using the ion-specific fluorescent probe, FluoZin-3-AM™ (FluoZin-3, Molecular Probes, Inc.). Exponential phase cultures were treated with control 5% mannitol vehicle or Zn(OAc)₂ in the presence or absence of MGd as described above, for 4 hours. Following treatment, cells were isolated by centrifugation. Cell pellets were washed and re-suspended in a solution of 0.5% BSA in PBS. An aliquot of 10⁶ cells (200 μL) was removed, centrifuged, and treated with FluoZin-3 reaction buffer. An aliquot of the cell suspension was supplemented with 2 μg/mL propidium iodide (Sigma Biochemical), incubated for 5 minutes, and subjected to two-parameter flow cytometric analysis.

Results: The above findings indicated that MGd treatment might alter the cellular availability of zinc ion. Therefore, cultures of A549 cells were treated with control 5% mannitol vehicle or Zn(OAc)₂ in the presence or absence of MGd for 4 hours, and cells were analyzed for free (chelatable) intracellular zinc using the ion-specific dye, FluoZin-3. Treatment with 100 μM Zn(OAc)₂ significantly increased the cell-associated fluorescence of FluoZin-3 at 530 nm (FIG. 2C). Co-incubation of cultures with 50-100 μM Zn(OAc)₂ and MGd led to synergistic increases in the corresponding fluorescent signals. Next, we attempted to determine the mechanisms by which MGd treatment alters levels of free intracellular zinc. Cells were incubated in the complete absence of exogenous zinc (serum-free medium) to evaluate the role zinc uptake plays in this process. Cells were also treated with actinomycin D to minimize the expression of genes (i.e. metallothionein family members and ZnT1) that might mask drug-induced changes in free intracellular zinc levels. Interestingly, co-treatment of cells with MGd and actinomycin D lead to a 1.5-fold increase in cellular fluorescence (inset to FIG. 2C). No increase in cellular fluorescence at 530 nm was observed in the absence of FluoZin-3 in these experiments.

### Example 7-Example 10

### Effect of MGd on the Antiproliferative Effects of Zinc Acetate

Methods: The proliferation of exponential phase cultures was assessed by colorimetric assay. In brief, 2×10⁵ suspension cells per well were seeded on 96-well V-bottom microtiter plates. Stock solutions of control vehicle, MGd or Zn(OAc)₂ in medium were added and plates were incubated at 37° C. under a 5% CO₂/95% air atmosphere. After 24 hours, medium was replaced with fresh medium. After 2 additional days, medium was exchanged with fresh medium (150 μL/well) supplemented with 3-(4,5-dimethylthiazol-2-yl)-2,5-diphenyltetrazolium bromide (MTT, 0.5 mg/mL, Sigma Biochemical, St. Louis, Mo.). Plates were incubated at 37° C. and viable cells measured.

Results: To determine whether MGd modulation of metal ion availability was restricted to quiescent cell cultures, exponential growth phase A549 cultures were treated with zinc in the presence or absence of MGd for 24 hours. Following an additional 48 hr growth period, viable cells were measured using a tetrazole reduction assay. Zinc treatment modestly inhibited A549 cell proliferation at the highest (100 μM) concentration. Treatment with MGd alone had no effect on proliferation, but enhanced the anti-proliferative activity of zinc at all MGd concentrations tested (FIG. 2D).

### Example 8

### MGd Modulation of Zinc Activity in PC3 Prostate Cancer Cells and Ramos Lymphoma Cells

Methods: Cellular Viability—Cell viability was determined by using propidium iodide (PI) flow cytometric analysis. Cells from plateau phase cultures grown in T-25 flasks were harvested as described above, except that cells present in the growth medium and wash solution were isolated by centrifugation and included in the analysis. Cells were resuspended in 1 mL PBS, an aliquot of 3×10⁵ cells transferred to a 4 mL tube, and the cells isolated by centrifugation. Cell pellets were resuspended in PBS supplemented with 2 μg/mL propidium iodide (Sigma), incubated for 5 minutes at ambient temperature, and subjected to flow cytometric analysis. Flow cytometry was performed on a FACSCalibur instrument and data were analyzed using the CellQuest Pro software package (BD Biosciences).

Results: Treatment of plateau phase PC3 cultures with zinc had little effect on cell viability after 24 hr (data not shown), but increased cell death (2-fold at 100 μM zinc), as measured by propidium iodide exclusion, was apparent within 48 hr (FIG. 3A). MGd enhanced the cytotoxic effect of zinc (e.g., 5-fold at 100 μM zinc), similar to what was observed in the A549 line. Metallothionein and ZnT1 levels were elevated following treatment with MGd and zinc (FIG. 3B). As before, treatment with MGd led to increased zinc-associated cellular fluorescence in the presence of exogenous zinc (FIG. 3C). Indeed, in this cell line, incubation with MGd alone led to a more marked increase in cellular fluorescence after 4 hours (ca. 2-fold) than in A549. Interestingly, PC3 cell proliferation was also inhibited by zinc (FIG. 3D). As in the A549 line, treatment with MGd enhanced the effect of zinc. This effect was confirmed by colony forming assay. A surviving fraction of 0.16 was measured in the presence of 10 μM MGd and 75 μM zinc, whereas zinc or MGd alone was without effect (data not shown).

Furthermore, we examined the effect of MGd and zinc treatment on viability, metallothionein and ZnT1 gene expression, and intracellular levels of free zinc in Ramos B-cell lymphoma cells grown in suspension (FIGS. 4A-C). Overall, the results were qualitatively similar to those obtained with A549 and PC3, with the difference that changes were generally observed at lower (i.e., 25-50 μM) concentrations of zinc. Cellular proliferation was strongly inhibited by 50 μM zinc and MGd, whereas either agent alone was without effect (FIG. 4D).

### Example 9

### Inhibition of Lipoate Reduction

Methods: Lipoate Reduction. Thioredoxin reductase activity was assessed by measuring the rate of lipoate reduction. In brief, A549 or PC3 cells (10,000 cells/well) were plated on 96-well plates and allowed to adhere overnight and grow two additional days until confluent. Cells were treated with MGd, zinc, or 5% mannitol for 2-4 hours, as indicated. Medium was removed, cells were washed with Hanks Balanced Salt Solution (HBSS), and a solution of 5 mM lipoic acid and 1 mM 5,5′-dithiobis(2-nitrobenzoic acid) in HBSS (100 μL/well) was added. Plates were incubated at ambient temperature in the dark. At chosen time intervals, plate absorbance was measured at 405-650 nm. Plate absorbances were normalized to wells containing neither exogenous zinc nor texaphyrin complex to allow plate-to-plate comparison. In some experiments, L-buthionine-[S,R]-sulfoximine (BSO, 100 μM) was added 24 hours prior to MGd or zinc treatment, to inhibit any contribution to lipoate reduction made by glutathione-dependent pathways (28). Actinomycin D (2.5 μg/mL) or cycloheximide (10 μg/mL), where present, was added 1 hour prior to texaphyrin or zinc treatment. Viability was checked on parallel plates using the tetrazolium dye 3-(4,5-dimethylthiazol-2-yl)-5-(3-carboxymethoxyphenyl)-2-(4-sulfophenyl)-2H-tetrazolium (MTS) following the manufacturers protocol (Promega Corp.). MTS signal was not altered by any treatment condition within the time frame of the lipoate reduction assay.

Results: An investigation of the effect of MGd treatment on the complementary, glutathione-independent thioredoxin pathway was carried out. Thioredoxin reductase activity can be evaluated in intact cells by measuring the reduction of the oxidized form of the cell-permeable cofactor lipoate to its reduced form, dihydrolipoate. Using this method, we observed a modest (approximately 10%) inhibition of lipoate reduction in plateau phase A549 cultures following a two-hour treatment with MGd alone (FIG. 5A). Moreover, up to a 30% inhibition of lipoate reduction was observed following treatment of cultures with zinc alone, consistent with literature reports of thioredoxin reductase inhibition by this cation. Greater inhibition (up to 60%) was observed following treatment with both zinc and MGd (FIG. 5A). This effect of MGd was dose-dependent, with an apparent inhibitory concentration of approximately 2.5 μM. The inhibition of lipoate reduction was less pronounced (approximately 30%) after 4 hours of incubation (FIG. 5B). Pre-treatment with either actinomycin D (FIG. 5C) or cycloheximide (data not shown) restored the inhibitory effect of both MGd and zinc on lipoate reduction. These findings lead us to suggest that A549 cells compensate for increases in intracellular zinc levels by transcription and translation-dependent processes. Lipoate reduction was also inhibited in cells treated with zinc 1-hydroxypyridine-2-thiol, a zinc ionophore that increases the concentration of intracellular zinc (FIG. 5D). Pre-incubation of cultures with the glutathione synthesis inhibitor BSO increased the degree of inhibition by MGd and zinc, demonstrating that both agents were targeting the glutathione-independent lipoate reduction pathway (data not shown). Similar observations were obtained using plateau phase PC3 prostate cancer cell cultures (FIGS. 5E-H). However, lipoate reduction was inhibited at lower concentrations of zinc in PC3 cells as compared to A549. Lipoate reduction was similarly inhibited by MGd and zinc in exponential Ramos lymphoma cultures.

### Example 10

### Intracellular Free Zinc is Elevated in Ramos Cells Treated with MGd and Zinc

Methods: Intracellular Free Zinc. The concentration of intracellular free zinc was assessed using the ion-specific fluorescent probe, FluoZin-3-AM™ (FluoZin-3, Molecular Probes, Inc.). Exponential phase cultures were treated with control 5% mannitol vehicle or Zn(OAc)₂ in the presence or absence of MGd as described above, for 4 hours. Following treatment, cells were isolated by centrifugation. Cell pellets were washed and re-suspended in a solution of 0.5% BSA in PBS. An aliquot of 10⁶ cells (200 μL) was removed, centrifuged, and treated with FluoZin-3 reaction buffer. An aliquot of the cell suspension was supplemented with 2 μg/mL propidium iodide (Sigma Biochemical), incubated for 5 minutes, and subjected to two-parameter flow cytometric analysis as described previously.

Results: Co-treatment of Ramos cells with 10 μM MGd and 50 μM zinc led to a 4-fold increase in median FluoZin-3 fluorescence within 2 hours compared to control cells (FIG. 6A). This signal increased to approximately 8-fold within 12 hours, and remained constant thereafter. Treatment with 10 μM MGd or 50 μM zinc alone led to small increases in FluoZin-3 fluorescence within 2 hours, which returned to baseline levels by 12 hours. As a negative control, we found no increase in cellular fluorescence at 530 nm in the absence of FluoZin-3.

### Example 11

### MGd and Zinc Increase Oxidative Stress in Ramos Cells

Methods: Reactive oxygen species were measured in live cells as intracellular peroxides by monitoring the oxidation of 2′,7′-dichlorofluorescin-diacetate (DCFA) to 2′,7′-dichlorofluorescein (DCF) (Molecular Probes). Cells (1×10⁶ per mL) were incubated in a solution of 1 μg per mL DCFA in 0.5% BSA in HBSS for 15 minutes at 37° C. Two mL additional 0.5% BSA in HBSS was added, cells were isolated by centrifugation, and the pellet re-suspended in a solution of 50 μg/mL 7-aminoactinomycin D (7-AAD) in 0.5% BSA in HBSS. Cell suspensions were incubated at ambient temperature for 2 to 3 minutes, and stored on ice until analysis. The fluorescent intensity in live (i.e., 7-AAD impermeable) cells was analyzed by flow cytometry.

Results: Oxidative stress in Ramos cells treated with MGd and/or zinc was measured over time by monitoring the conversion of dichlorofluorescin acetate (DCFA) to dichlorofluorescein (DCF) (FIG. 6B). Cultures of Ramos cell treated with both 10 μM MGd and 50 μM zinc displayed 1.8-fold increase in median DCF signal within 2 hours, which gradually diminished over the 24 hour time course of the experiment to background levels. Treatment with MGd or zinc alone also increased DCF signal, albeit not to the same degree as the combination. Treatment of Ramos cells with hydrogen peroxide also increased DCF and FluoZin-3 fluorescence.

### Example 12

### MGd and Zinc Treatment Leads to Apoptosis in Ramos Cells

Methods: Annexin V/PI. Cells from exponential phase cultures were treated with MGd, zinc, or control (5% mannitol) solution for 24 or 48 hours. After incubation, cells were harvested and washed twice with a solution of 0.5% bovine serum albumin (BSA) in Hanks buffered saline (HBSS). An aliquot of cells (1×10⁶) was added to 500 μL diluted binding buffer from the ANNEXIN-V PI Kit (BD Biosciences, San Jose, Calif.). Cells were pelleted, re-suspended in 100 μL of diluted binding buffer, and treated with the ANNEXIN-V PI reagent as per the manufacturer's protocol. Flow cytometry was performed on a FACSCalibur instrument and data were analyzed using the CellQuest Pro software package (BD Biosciences).

Mitchondrial membrane potential. Loss of the mitochondrial membrane potential (ΔΨₘ) of cells was measured by the use of JC-1 (Molecular Probes, Inc., Eugene, Oreg.). Cells undergoing early apoptosis lose fluorescence in the 585 nm channel and gain it in the 530 nm channel. Briefly, cells cultured as described above were washed twice with complete medium, re-suspended in 0.5 mL JC-1 solution (10 μg/mL in complete medium), and incubated at 37° C. for 15 minutes. Cells were isolated by centrifugation, washed once and then re-suspended in 0.5 mL solution of 0.5% BSA in PBS and assayed immediately on the flow cytometer.

Results: To determine the rate at which co-treated Ramos cells undergo cell death, Ramos cultures treated as above were analyzed using fluorescein (FITC)-labeled Annexin-V reagent to detect early and late apoptotic events (FIG. 6C). In addition, the dye JC-1 was employed to assess mitochondrial function (FIG. 6D). In cultures treated with MGd and zinc, 21% of Ramos cells exhibited a positive Annexin-V signal within 8 hours of treatment. This fraction increased to 30% within 12 hours and 68% by 24 hours. Analogous results were obtained using JC-1, with 38% of cells exhibiting non-aggregated (green) JC-1 fluorescence characteristic of mitochondrial dysfunction within 8 hours of combined treatment with MGd and zinc. This fraction increased to 52% by 12 hours and 74% by 24-hours. No significant change in Annexin-V signal or JC-1 fluorescence was observed within 4 hours or as a result of treatment with MGd or zinc alone.

### Example 13

### MGd and Zinc Treatment Leads to Cell Cycle Arrest in Ramos Cells

Methods: Cell Cycle Analysis—Exponential phase cultures were treated with control 5% mannitol vehicle or Zn(OAc)₂ in the presence or absence of MGd as described above. Thirty minutes prior to harvest, cultures were treated with 5-bromo-2′-deoxyuridine (BrdU) at a final concentration of 10 μM. Cells were isolated by centrifugation, washing once with 0.5% BSA in PBS, and the resulting cell pellets fixed using 0.5 mL Cytofix/Cytoperm reagent (BD Biosciences). After incubation at ambient temperature for 30 minutes, cells were isolated by centrifugation, washed with 3% FBS in PBS, re-suspended in 10% DMSO in medium, and stored at −20° C. until analysis. Cells were stained using a fluorescein-conjugated anti-BrdU antibody (clone PRB1, E-Bioscience, San Diego, Calif.) and 7-AAD. Cell cycle occupancy was analyzed by flow cytometry using fluorescein signal as a measure of DNA synthesis and 7-AAD signal as a measure of DNA content. For comparison, cultures were treated with 5-fluoro-2′-deoxyuridine, hydroxyurea, or irradiated using a ¹³⁷Cs irradiator (Model 40 Gammacell, J. L. Shepherd & Assoc., San Fernando, Calif.).

Mitchondrial membrane potential. Loss of the mitochondrial membrane potential (ΔΨₘ) of cells was measured by the use of JC-1 (Molecular Probes, Inc., Eugene, Oreg.). Cells undergoing early apoptosis lose fluorescence in the 585 nm channel and gain it in the 530 nm channel. Briefly, cells cultured as described above were washed twice with complete medium, re-suspended in 0.5 mL JC-1 solution (10 μg/mL in complete medium), and incubated at 37° C. for 15 minutes. Cells were isolated by centrifugation, washed once and then re-suspended in 0.5 mL solution of 0.5% BSA in PBS and assayed immediately on the flow cytometer.

Annexin V/PI. Cells from exponential phase cultures were treated with MGd, zinc, or control (5% mannitol) solution for 24 or 48 hours. After incubation, cells were harvested and washed twice with a solution of 0.5% bovine serum albumin (BSA) in Hanks buffered saline (HBSS). An aliquot of cells (1×10⁶) was added to 500 μL diluted binding buffer from the ANNEXIN-V PI Kit (BD Biosciences, San Jose, Calif.). Cells were pelleted, re-suspended in 100 μL of diluted binding buffer, and treated with the ANNEXIN-V PI reagent as per the manufacturer's protocol. Flow cytometry was performed on a FACSCalibur instrument and data were analyzed using the CellQuest Pro software package (BD Biosciences).

Results: In order to examine the kinetics of growth rate responses to co-treatment with MGd and zinc acetate. To do this, Ramos cultures co-treated as above were labeled with 5-bromo-2′-deoxyuridine (BrdU) and 7-aminoactinomycin D (7-AAD) in order to determine cell cycle occupancy. As shown in FIG. 7, co-treatment with MGd and zinc halted BrdU incorporation in S-phase cells actively synthesizing DNA within 8 hours. It also led to inhibition of cell entry and progression through G1 and G2/M phases (as determined by DNA content analysis, data not shown). The effects of treatment with 5-fluoro-2′-deoxyuridine, hydroxyurea, and ionizing radiation are shown for comparison.

### Example 14

### MGd Modulation of Zinc Activity in Other Lymphoma Cell Lines

Methods: Annexin V/PI. Cells from exponential phase cultures were treated with MGd, zinc, or control (5% mannitol) solution for 24 or 48 hours. After incubation, cells were harvested and washed twice with a solution of 0.5% bovine serum albumin (BSA) in Hanks buffered saline (HBSS). An aliquot of cells (1×10⁶) was added to 500 μL diluted binding buffer from the ANNEXIN-V PI Kit (BD Biosciences, San Jose, Calif.). Cells were pelleted, re-suspended in 100 μL of diluted binding buffer, and treated with the ANNEXIN-V PI reagent as per the manufacturer's protocol. Flow cytometry was performed on a FACSCalibur instrument and data were analyzed using the CellQuest Pro software package (BD Biosciences).

Cellular Proliferation—The proliferation of exponential phase cultures was assessed by colorimetric assay. In brief, 2×10⁵ suspension cells per well were seeded on 96-well V-bottom microtiter plates. Stock solutions of control vehicle, MGd or Zn(OAc)₂ in medium were added and plates were incubated at 37° C. under a 5% CO₂/95% air atmosphere. After 24 hours, medium was replaced with fresh medium. After 2 additional days, medium was exchanged with fresh medium (150 μL/well) supplemented with 3-(4,5-dimethylthiazol-2-yl)-2,5-diphenyltetrazolium bromide (MTT, 0.5 mg/mL, Sigma Biochemical, St. Louis, Mo.). Plates were incubated at 37° C. and viable cells measured.

Intracellular Free Zinc—The concentration of intracellular free zinc was assessed using the ion-specific fluorescent probe, FluoZin-3-AM™ (FluoZin-3, Molecular Probes, Inc.). Exponential phase cultures were treated with control 5% mannitol vehicle or Zn(OAc)₂ in the presence or absence of MGd as described above, for 4 hours. Following treatment, cells were isolated by centrifugation. Cell pellets were washed and re-suspended in a solution of 0.5% BSA in PBS. An aliquot of 10⁶ cells (200 μL) was removed, centrifuged, and treated with FluoZin-3 reaction buffer. An aliquot of the cell suspension was supplemented with 2 μg/mL propidium iodide (Sigma Biochemical), incubated for 5 minutes, and subjected to two-parameter flow cytometric analysis.

Results: The effect of MGd and zinc on proliferation was examined in Ramos and several other hematologic cell lines (see supplemental data). The B-cell lines (Ramos, Raji, DB, DHL-4 and HF-1) appeared to be more sensitive than the Jurkat T-cell line and the myeloid cell lines K562 and HL-60. In all cell lines, sensitivity to zinc was increased by MGd, whereas low concentrations of zinc or MGd alone had no significant effect.

The five B-cell lines were further tested for changes in intracellular free zinc levels and oxidative stress after 4 hours, and apoptosis after 24 and 48 hours of treatment with 10 μM MGd and 50 μM zinc acetate (FIG. 8). Increases in FluoZin-3, DCF, and Annexin-V-FITC fluorescence relative to control were in the following order: Ramos>DHL-4>DB>Raji,>HF-1. This roughly matches the sensitivity of these lines to treatment with zinc in the proliferation assay. However, there was no significant change in median DCF fluorescence in the DB or HF-1 lines at four hours. No changes in FluoZin-3, DCF, or Annexin-V-FITC fluorescence were observed in Jurkat, K562, or HL-60 lines under these conditions.

### Example 15

### Gene Expression Profiling of MGd-Treated Ramos Cells

Methods: Gene Expression Profiling—MGd (10 μM), Zn(OAc)₂ (25 or 50 μM, the combinations, or control (5% mannitol) solution were added to Ramos cultures. Each treatment was performed in triplicate. After 4 hours of incubation, all cultures were washed twice with 0.5% BSA in HBSS and total RNA was isolated and subjected to analysis on Human Genome U133 Plus 2.0 Arrays (Affymetrix, Santa Clara, Calif.). Microarray Suite version 5.0 software (Affymetrix) was used to generate raw gene expression scores and normalize the relative hybridization signal from each experiment. All gene expression scores were set to a minimum value of fifty in order to minimize noise associated with less robust measurements of rare transcripts. Both the parametric Student's t-test and the permutation-based significance analysis of microarrays (SAM) were used to determine genes differentially expressed in treatment versus control groups. We report data from genes that are at least 1.5-fold differentially expressed relative to controls using the Student's t-test (p≦0.005) since this empirically proved to be a more stringent criterion than SAM analysis using the same 1.5-fold cut-off and a <1% False Discovery Rate (FDR).

Results: To assess the effects of MGd or zinc treatment on gene expression profiles, total cellular RNA was isolated from exponential phase Ramos cultures treated in triplicate with control vehicle (5% mannitol), 10 μM MGd, 25 or 50 μM zinc acetate, or the zinc and MGd combinations for 4 hours and analyzed on oligonucleotide microarrays. Twenty-nine transcripts (twenty-five of which had annotated functions) showed differential expression in response to MGd treatment that reached our criteria for statistical significance (A 0.5-fold, p≦0.005) (FIG. 11). The most prominent consequence of MGd treatment was the up-regulation of MTF-1 regulated genes, including metallothionein and zinc transporter family transcripts, as was observed previously in A549 lung cancer plateau phase cultures. The levels of six transcripts were down-regulated, including SLC39A10 which encodes a transporter involved in the uptake of zinc. Interestingly, a splice variant of this transporter was significantly increased, presumably reflecting additional mechanisms operating to regulate levels of intracellular free zinc. In addition, we observed HIF-1 related transcripts displaying significant changes including DDIT4, EGLN1, and PFKFB3 (FIG. 11). Similar expression patterns were observed in response to 50 μM zinc.

Depending on treatment condition, the number of significantly changed transcripts was: 25 μM zinc acetate (3)<MGd alone (29)<MGd+25 μM zinc acetate (278)˜50 μM zinc acetate (347)<<MGd+50 μM zinc acetate (1,226). While only one annotated transcript (SLC39A10) was differentially regulated in response to treatment with 25 μM zinc acetate, 347 transcripts were differentially expressed in response to treatment with the higher concentration of 50 μM zinc acetate (284 up- and 63 down-regulated). A total of 12/29 (41%) of the transcripts significantly changed by MGd treatment were also changed (≧1.5-fold in the same direction, p≦0.005) by treatment with 50 μM zinc acetate (FIG. 9A). However, 28/29 (97%) MGd-responsive genes were also differentially expressed in the same direction in Ramos cultures treated with 50 μM zinc using less stringent criteria (≦1.2-fold, p≦0.05).

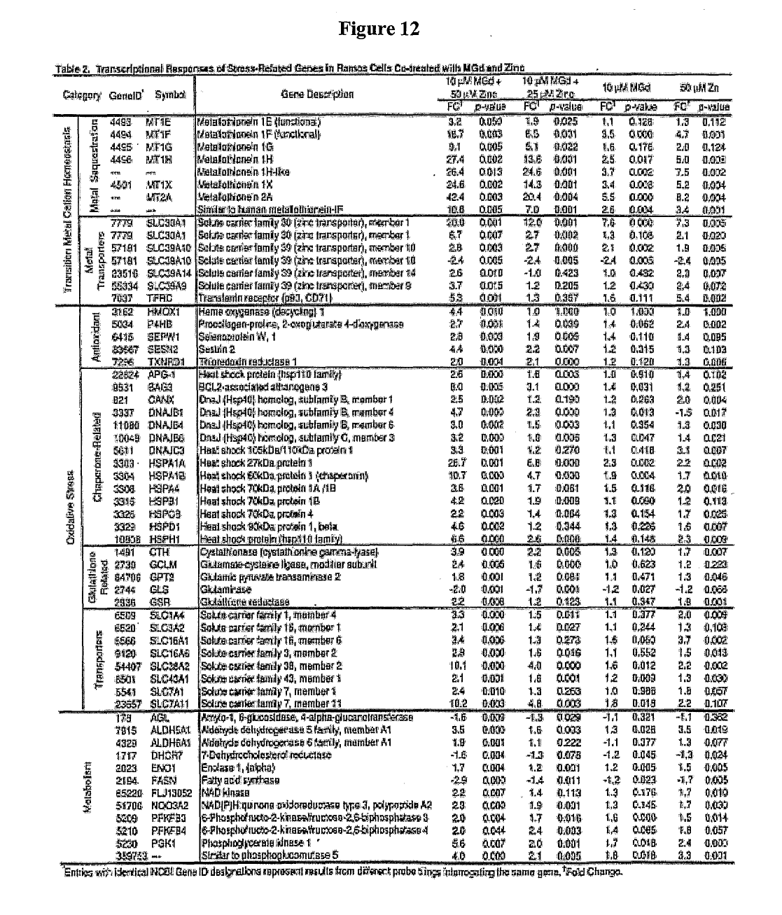

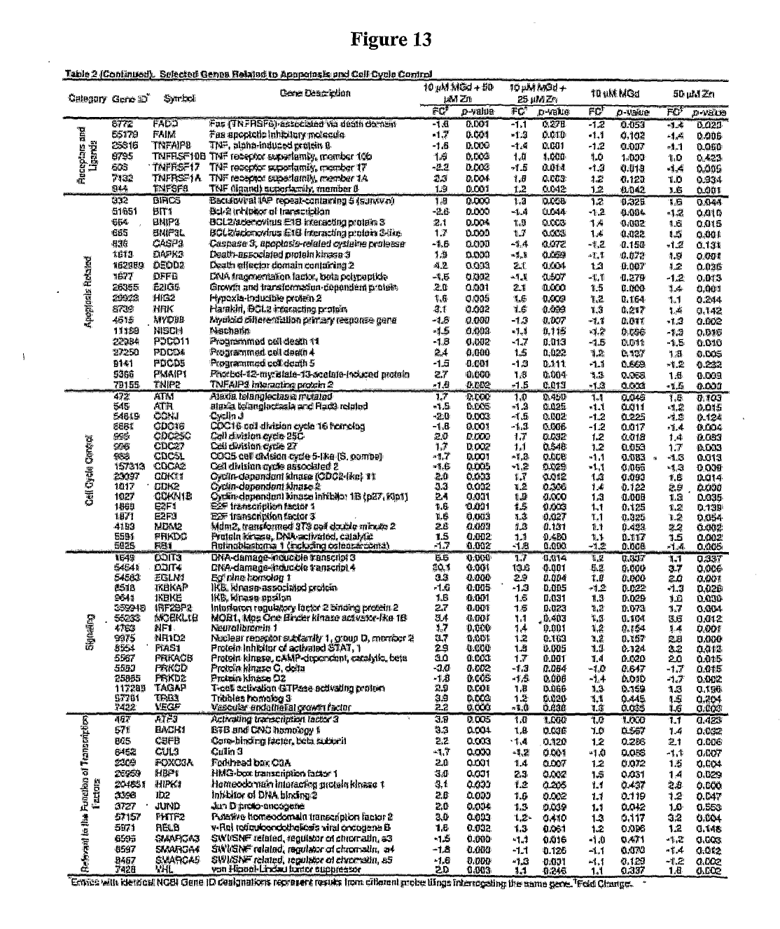

To simplify presentation and interpretation, a selected group of the 1,226 differentially expressed transcripts in the 10 μM MGd+50 μM zinc acetate group are shown in FIG. 12. A total of 64% (178/278) of the differentially expressed transcripts in the 10 μM MGd and 25 μM zinc acetate group were shared with the 10 μM MGd and 50 μM zinc acetate group (FIG. 9B). Using less stringent criteria (1.2-fold, p≦0.05), 253/278 (91%) of the transcripts differentially expressed in the former group were shared with the latter group. This is especially interesting given that changes in cell viability were observed in the 10 μM MGd and 50 μM zinc acetate group but not in the 10 μM MGd and 25 μM zinc acetate group. Overall, we observed a trend towards larger magnitudes of differential gene expression in response to co-treatment with MGd and zinc relative to individual treatments (FIG. 10-12).

### Example 16

### Levels of HIF-1α Metallothioneins, and Heme Oxygenase-1

Methods: Total HIF-1α protein was detected by sandwich ELISA using the DuoSet IC™ HIF-1α ELISA kit obtained from R&D Systems (Minneapolis, Minn.). All incubations were carried out at ambient temperature. Briefly, 96-well plates were coated with HIF-1α capture antibody overnight prior to blocking with 5% BSA in wash buffer. Protein lysates (50 μg protein per well prepared according to the manufacturer's instructions) were added for 2 hours, whereupon plates were washed and a biotinylated detection antibody specific for HIF-1α was added. A streptavidin-horseradish peroxidase format was used for detection. The optical density at 450 minus 570 m was measured using a microplate reader (SpectraMax Plus, Molecular Devices, Palo Alto, Calif.). HIF-1α concentrations were calculated by linear regression using a standard curve prepared from HIF-1α standard supplied with the ELISA kit.

Results: To demonstrated that some of the transcriptional changes determined by DNA microarray analysis were also reflected in alterations of protein expression, cellular levels of HIF-1α in Ramos cells were measured by ELISA following treatment with MGd and zinc for four hours (FIG. 9C). Total cellular HIF-1α levels were increased 1.5 to 3-fold by treatment with MGd, zinc, or the combination. As expected, HIF-1α levels were also increased by treatment with cobalt acetate or by the use of hypoxic culture conditions. Levels of metallothioneins (MT) and heme oxygenase 1 (HO-1) were shown by Western blot to be increased following co-treatment with MGd and zinc for 16 hours (FIG. 9D). MT and HO-1 are proteins with expression induced by MTF-1 and NRF-2, respectively.

What is claimed is:

1. A composition comprising an amount of a metal-containing texaphyrin sufficient to cause a reduction in thioredoxin reductase activity of between about 10 to about 90%.

2. The composition of claim 1 wherein the reduction in thioredoxin reductase activity is at least about 30%.

3. (canceled)

4. (canceled)

5. (canceled)

6. (canceled)

7. The composition of claim 1 further comprising an amount of a zinc (II) reagent sufficient to cause a reduction in thioredoxin reductase activity of between about 10 to about 90%.

8. The composition of claim 7 wherein the zinc (II) reagent is selected from the group consisting of zinc acetate, zinc chloride, zinc citrate, zinc lactate zinc gluconate, L-carnosine salt, zinc fetuin, zinc sulfate, zinc bacitracin, zinc seleno-bacitracin, chelated zinc, and zinc ionophores such as zinc 1-hydroxypyridine-2-thiol.

9. The composition of claim 8 wherein the zinc (II) reagent is zinc acetate.

10. (canceled)

11. (canceled)

12. (canceled)

13. (canceled)

14. (canceled)

15. (canceled)

16. (canceled)

17. (canceled)

18. (canceled)

19. (canceled)

20. A method for treating cancer comprising: administering to a patient having cancer an amount of a metal-containing texaphyrin sufficient to cause a reduction in thioredoxin reductase activity of between about 10 to about 90%.

21. The method of claim 20 wherein the reduction in thioredoxin reductase activity is at least about 30%.

22. (canceled)

23. (canceled)

24. The method of claim 20 further comprising administering to the patient having cancer an amount of a zinc (II) reagent sufficient to cause a reduction in thioredoxin reductase activity of between about 10 to about 90%.

25. The method of claim 24 wherein the zinc (II) reagent is selected from the group consisting of zinc acetate, zinc chloride, zinc citrate, zinc lactate zinc gluconate, L-carnosine salt, zinc fetuin, zinc sulfate, zinc bacitracin, zinc seleno-bacitracin, chelated zinc, and zinc ionophores such as zinc 1-hydroxypyridine-2-thiol.

26. The method of claim 25 wherein the zinc (II) reagent is zinc acetate.

27. (canceled)

28. (canceled)

29. (canceled)

30. (canceled)

31. (canceled)

32. The method of claim 20 or claim 24 wherein the metal-containing texaphyrin is a compound of Formula III: 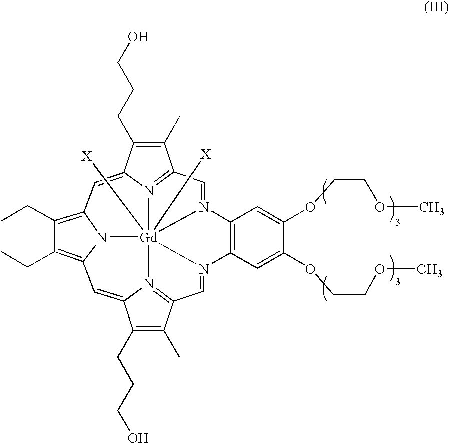
or a compound of Formula IV: 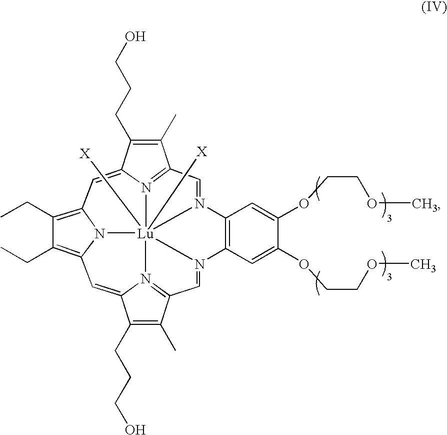
wherein X is independently selected from the group consisting of OH⁻, AcO⁻, Cl⁻, Br⁻, I⁻, F⁻, H₂PO₄⁻, ClO⁻, ClO₂⁻, ClO₃⁻, ClO₄⁻, HCO₃⁻, HSO₄⁻, NO₃⁻, N₃⁻, CN⁻, SCN⁻, OCN⁻; sugar derivatives, cholesterol derivatives, PEG acids, organic acids, organosulfates, organophosphates, phosphates or inorganic ligands; or X is derived from an acid selected from the group consisting of gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid.

33. (canceled)

34. A composition for treating cancer comprising an amount of a metal-containing texaphyrin and an amount of a zinc (II) reagent sufficient to cause an increase in a HIF-1α level of about 3.0 fold.

35. (canceled)

36. (canceled)

37. (canceled)

38. (canceled)

39. The composition of claim 34 wherein the zinc (II) reagent is selected from the group consisting of zinc acetate, zinc chloride, zinc citrate, zinc lactate zinc gluconate, L-carnosine salt, zinc fetuin, zinc sulfate, zinc bacitracin, zinc seleno-bacitracin, chelated zinc, and zinc ionophores such as zinc 1-hydroxypyridine-2-thiol.

40. The composition of claim 34 wherein the metal-containing texaphyrin is a compound of Formula III: 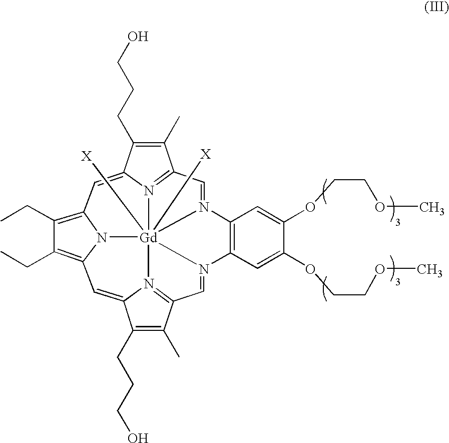
or a compound of Formula IV: 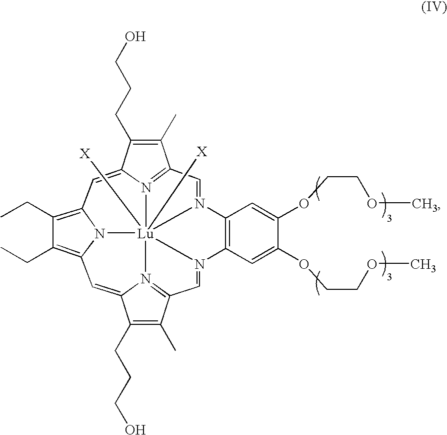
wherein X is independently selected from the group consisting of OH⁻, AcO⁻, Cl⁻, Br⁻, I⁻, F⁻, H₂PO₄⁻, ClO⁻, ClO₂⁻, ClO₃⁻, ClO₄⁻, HCO₃⁻, HSO₄⁻, NO₃⁻, N₃⁻, CN⁻, SCN⁻, OCN⁻; sugar derivatives, cholesterol derivatives, PEG acids, organic acids, organosulfates, organophosphates, phosphates or inorganic ligands; or X is derived from an acid selected from the group consisting of gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid.

41. (canceled)

42. (canceled)

43. A method for treating cancer comprising: administering to a patient having cancer an amount of a metal-containing texaphyrin and an amount of a zinc (II) reagent sufficient to cause an increase in a HIF-1α level of about 3.0 fold.

44. The method of claim 43 wherein the zinc (II) reagent is selected from the group consisting of zinc acetate, zinc chloride, zinc citrate, zinc lactate zinc gluconate, L-carnosine salt, zinc fetuin, zinc sulfate, zinc bacitracin, zinc seleno-bacitracin, chelated zinc, and zinc ionophores such as zinc 1-hydroxypyridine-2-thiol.

45. The method of claim 43 wherein the metal-containing texaphyrin is a compound of Formula III: 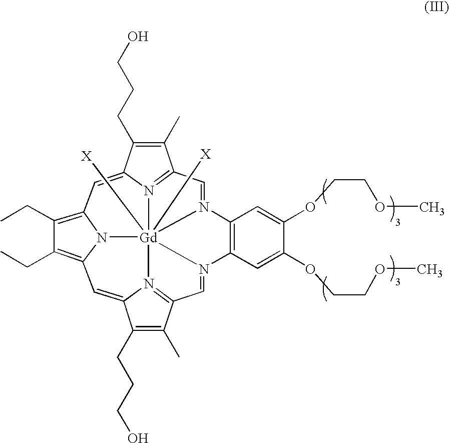
or a compound of Formula IV: 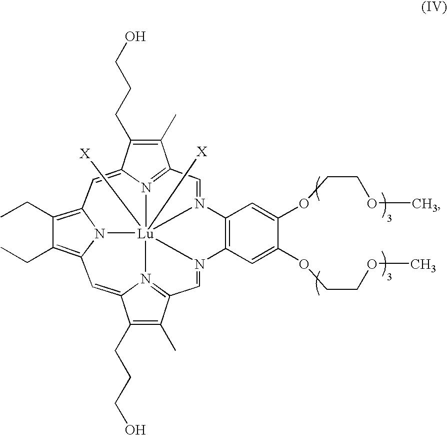
wherein X is independently selected from the group consisting of OH⁻, AcO⁻, Cl⁻, Br⁻, I⁻, F⁻, H₂PO₄⁻; ClO⁻, ClO₂⁻, ClO₃⁻, ClO₄⁻, HCO₃⁻, HSO₄⁻, NO₃⁻, N₃⁻, CN⁻, SCN⁻, OCN⁻; sugar derivatives, cholesterol derivatives, PEG acids, organic acids, organosulfates, organophosphates, phosphates or inorganic ligands; or X is derived from an acid selected from the group consisting of gluconic acid, glucoronic acid, cholic acid, deoxycholic acid, methylphosphonic acid, phenylphosphonic acid, phosphoric acid, formic acid, propionic acid, butyric acid, pentanoic acid, 3,6,9-trioxodecanoic acid, 3,6-dioxoheptanoic acid, 2,5-dioxoheptanoic acid, methylvaleric acid, glycolic acid, pyruvic acid, oxalic acid, malic acid, malonic acid, succinic acid, maleic acid, fumaric acid, tartaric acid, citric acid, methanesulfonic acid, ethanesulfonic acid, benzoic acid, salicylic acid, 3-fluorobenzoic acid, 4-aminobenzoic acid, cinnamic acid, mandelic acid, and p-toluene-sulfonic acid.

46. A method for predicting treatment efficacy comprising: monitoring oxidative stress related genes in plasma or target cells of an animal subject bearing a tumor, a neoplastic disease or atheroma prior to and/or after treatment with a metal-containing texaphyrin and/or a zinc (II) reagent; wherein the monitoring is a modulation of administration of the treatment with the metal-containing texaphyrin and/or the zinc (II) reagent.

47. The method of claim 46 wherein the modulation of administration of the treatment further includes: administering a therapeutic agent or a ionization radiation; adjusting the dosage of the metal-containing texaphyrin and/or the zinc (II) reagent; route of administration of the metal-containing texaphyrin and/or the zinc (II) reagent, frequency of administration of the metal-containing texaphyrin and/or the zinc (II) reagent; type of carrier of the metal-containing texaphyrin and/or the zinc (II) reagent; duration of treatment with the metal-containing texaphyrin and/or the zinc (II) reagent; enantiomeric form of the metal-containing texaphyrin and/or the zinc (II) reagent; crystal form of the metal-containing texaphyrin and/or the zinc (II) reagent; administering a fragment, analog, or variant of the metal-containing texaphyrin and/or the zinc (II) reagent; or a combination thereof.

48. (canceled)

49. (canceled)

50. (canceled)

51. (canceled)

52. (canceled)

53. (canceled)

54. (canceled)

55. (canceled)

56. (canceled)

57. (canceled)
